SEQUENCE LISTING (10 sequences)

SEQ ID NO: 1 - DNA, 9603 bp, Homo sapiens
ttcagtattgtgtagtacagaaaagatcacacatgtatatgtcagaagtagatgcacatgttcttttggctttttttctttgttttattcttttttcattttttgtgttttttgttagtttgttttgaattttttttcttgatgcatatgttccttcaacttattcagtacttccatgtgcagggctctggactagactttcaggatctaaggctgaatcctacacagtcctccctctgggaaagtgatgaaacctgtgagctctcctagcaactctgcagtcttccttcaagggcctgtgcttccttcagagactcacacaagaggtttatcatgagaaggaccgcactatttcatttcactcctacaagttttcatttacgttacacattgagaaagttatgagaagcaactgtcactctctggaggtggagactgccgtgattcacaaagacaagaggtaattttcctgtaatcacaggatgactcaggatggatcatgaagccgcccagctggagaagcagcatgtgcacaatgtgtacgagagcacagccccttacttcagcgacctgcagagcaaagcctggcctcgtgtccgccagttcctgcaagagcagaagccaggcagcctcatcgctgacataggttgtgggactggaaaatatcttaaagtgaacagccaggtacataccgtgggctgtgactactgtgggccactggtagagattgcccggaatagaggatgtgaagccatggtatgtgacaaccttaatctcccctttagggatgagggcttcgatgccatcatctccataggagtcatacatcatttttctacaaaacaaagaagaatcagagcaataaaagaaatggccagggtcttagttcccggaggccaactgatgatttacgtttgggcaatggaacaaaagaaccgtcactttgagaagcaagacgtgcttgttccatggaacagggctctgtgttcccagctcttctcagagtccagccagtctgggaggaagaggcagtgtggatacccagaaagaggccatccctaccatcctccttgctctgagtgtagctgttctgtttgttttaaagagcagtgtggttcaaaacggtcccacagcgtgggctatgaacctgctatggcaagaacctgttttgcaaatatttctaaggaaggcgaggaagaatatggattttacagcacattaggaaaatcgtttcgttcctggtttttctccagatctttggatgaatcgactctgaggaagcaaattgaaagagtaagacccttgaaaaacacagaagtttgggccagtagcactgtaacagtccagccttccagacactctagtttagactttgatcaccaagagccattttcaacaaaagggcaaagtttagatgaggaagtgtttgtggaatcttcttctggaaaacacttggagtggctgagagcaccaggcactctgaaacatttaaatggagaccatcaaggggaaatgaggagaaatggagggggaaattttctggatagcactaatactggtgtgaattgtgtggatgcaggcaacatagaagatgataatccttctgctagtaaaatattgagaaggatttctgcagttgattccacagatttcaacccagatgatacaatgtctgtcgaagatccacagactgatgttttggactccacagcctttatgcgctactaccatgtgtttcgagaaggggagctctgcagtctgctcaaggagaatgtgtcagagctccgtatcctgagttctgggaatgatcatggtaactggtgtatcattgcagagaaaaagagaggttgtgattgattggatccttttagacaactcctccaaaagatgaaccacattctttcctcttggtttgatatggttacctgaatttgcattcagtgttatttgttaatccatttacgctttggtctgcagagactattaattatttggttgttttgttttcatttttgaataagcacagattctggcattgaaagcacttgacaaagggtatttgtgcttaaatgttaatatacaagatctgaagaagcaacagaaagtacccttcagtacacctcagacttttttttaaccccagagagataaaatacatgtatagtgtttttcagtattacacattgatttaaaaagattatgctgttaaataatcttttaaaacggtatttttataaagtgaggggataatttctggttctcaggttataactgagagcagtgtgcaagataataggtaaatttgatccattgcacagatatactttgaaccatgtgatgagttatcttgttgccaaggccttgcttctacttaaagttttcagaaaactgagtgacagtggagagaaacaagaagttttacaaggactttactaaattataagcaaacttgcttcaaaataagttgacatgtgataataaggttttcaatgtagcccaggaggtttttaaaggcactgttaggctgagcatggtggctcatgcctgtaatcccagcactttgggaggccaaggcagaaggatcacttgaacccaggagttcaagaatagcctgggcaacatagcaagaccccatctctataaaaattagcaagatgtggtggtgcatgcctgtcatccaagctactcaagagactgaggcaggaggatcacgtgagcccaggaattcaaggctgcagtgaactatgattgcatcactgcactgcagcctgggcaacatagcaagactctgtctcaaaataataataataataataataaaggcgttgttagcttgtaaggagtggagtatgtaggtagtaggagttatatgcaagtacccaagtggtattcttccaatcttattagaagcatgaatattcaagattgatattactattgcttattagcaagattgttatcaatcatgcttattagaaggatgaatatccaagaccaagattgactaatgatgagtctgcatcaagaactaggcatttcttctgagttgacggactctttaggaaaggagaatctaagtgaagcactgattttagctctgagaacaaacaaattaaggtacagcatagttagccttggtagaggtatgacttggatttgctgtatcctttaaaatagtatctgggcatttattttattgaaggtgactacattttattagttatattaggaatttaggtagaatcaacttctactgattacaggttgaatttctgtcactttgtagagaaacgaatagactggacactgtgtggtcactgtttagatttgcccatgggtctctttaaatctatgtcatggatcctgagacacaaatataattaagacaggtctagagacaggagaagcagaaataagttgacccaggagtacagtctcaagtagttcattaatgagaaaattgacatctgacaagagtctttttactttatgctggatgaaaatccaaatcttgttttattttttccactaaaagtgactaaaataataacgaatttcatttgttcttgggttcttttttcctttaatgattgtgctataacttaaaataatgatgttacttttgaacaaacttaaagaaatatttttaaagcgtatctgaaaacgattgatgtttataactctcttttggcttcaaaataagattgtgttatcaccattttggtagatgaggttgtctggtgaaaatgatgcatatgagttgtactgttcagtgtacatcctgcagtagtggatgattgaaaacatatataagtggagtataaattaaaaattaatttggtttcttctatttcttttttttttttttttttttgagacagagtctcgctctgtcgcccaggctggagtgcactggcgcgatctcggctcactgtaagctccgcctcccgggttcacgccatcctcctgcctcagcctcccaagtagctgggactactggcacccaccacgacgcccggctgtttttttgtatttttaggagagacggggtttcaccgtgttagccaggatggtctcgatctcctgacgtcgtgatccacccgcctcggccccccacagtgctgggattacaggcgtgagccaccgcgcccggcctgttccttttatttcttaattcaggacactaaaccatgactgcaagggatttccttggtaaaaagaaaagattctcagagtcaaaatggtcttacaactcgggcttgacggcctttgaattatgaatggattgttcctctctctgaagcctattgtcacatgggtttttaatcctggccttgctgctagaaatctgtgcttgaagtcctctctttctgctgctagcctaccagttaaaagtcaagacttggtggaactcagtttaccagactctttagcctttgagctaaactgtctgagcaacctcttagatgtgcacacaccactttgtatgaaagggttctctagaacggttctttggagagaaatattttcatgtacgtttgacaggggtgtaaataaagcatgctgactaataagtcttttactcttcatctaatgaacataagaatctatgcatccagatattattttgtatacaaatatttaatttggtgattgataatctctctttggggtagtcacatggaaagctcttttaaatttaacttccgcctttggattttttttaaaaagccattgaagagcaaaactaatgtaaacgtcttgatcatttaaaaagcttgcttgtcctcgaaaggaaacacaggtcatcagtgagtataaacgtagacagttgatttgtgaatgctgtcggcctcaacttgcttgatgatagattctactgacctagctggagtaatctgatcacttacttccttattaatactagatcacacagtgtctttcttattccttcttctttacttactggcatcagcacagagtcccactatctgaaatagaagggagagtttgggggtttattggaggagtcttaaaaacttggttggcagctgggtacgatgactcacacctgtactcccagcacttcaggaagctgaggtgggtggatcagctgaggtcaggagtttgagaccagcctgaccaacatggtgaaaccccgtctttattaaatacaaaaaattagccaggttggtggcgcatgcctgtaatcccagctacttgggaggctgaggcaggagaatcgcttgaacctgggaggcggaggttgcagtgagccgagattgtgccattgcactccaacctgggcaacaagagtgaaactccatctccaacaaaacaaaactttgctggcttcctgatgcctcactgtctatttgaggaattccacagaatcttcaaaggatttgggggaaagcgcattaacatggacaaaggatggaatcaaaataatgttatagtgagaatcattcaagcacctatttaaattttttccaattgccagtatagtgatgatatgacaccagcatatcaaagtaactaacaaactagctacacaaacgtcttggagtttggtttcgttctcttttctcatcatagatctccgtgcagaatagtgctaattcttatttctcgtttgcctttctatttccttccaaattctacatgccagtaattcctctgtcttttaagtgaccatcaattcaataggcaaaaatttggagtaatccagagaaaaaaccatccaaatataaaccagctaggaacatgaatgcccctgattattaatggccaaaaaaaaagcactggtggattttaaattaaattaatacatatatacgagtttggaggagaacagaagttctaactcagtacttgaacttggtgggggagggcacaggttaaatatgagctgtgagcccccagttttggaggaaggaggaaatgggaaccaaccaccagacaagcagctgcagtctaagaaaaacaaattagggcaggcctcagcatctctttcctattacatagaaaatctgaaatgagaatagctgtgaacttttctaaatatgcaacattcaactatcgcaaggacaaaaaaccaaacaccgcatgttctcactcataggtgggaattgaacaatgagaacacatggacacagtaaggggaacatcacacactgggacctgttgtgaccccgctgagggagttcctcttgccctctacccccggggcttgtatatagattataaatatataagggggaaaggggtgggggggaggggttgtgcggctggggcctcacttcccctccttccccttctcctggtcccctgtcccaggggctgtttgttaaaaaagagtaataaaggatttttgtttttaaaaaaagcaacgttcattaatttatatgcagtcctcaccacagccctccaagataactatacttattgttcccattttgcagatgaggaaaactgaggcagaaagaaattaaattgcttaaggttacacacatagtaggtatcacacatttagttagaggcagagctgggattcaaacccaggtctggctcctagtctctgctcttaaccacaacaccatatactcccacccctgggcccagctcatgtgcttggcggagtattagagcctttcaaggtaagtgtgattatcccctttccaaatgagctttggaaaagtttaagcaaagattacacagctagacaataattcaatagtagtctcttacatattttcccttttatctttttttcatttgtaaatctgtttcgatttgatttgaattaaaaaatagttcttggttgcaaaatattctgggaccacttttctccaactcttcaacctttcaacaattatattattgttttcccattttataagtaaggcaactgaggactattcagatatttcattcactccatttgtttactaagccctgctgtgtgctggatacacagtctgctgtgtgctggatacatatgatacaaggtaatgactatctggcatcttgaaggtacccagaatattagaattaactgacccacaggtgccaagctgaaacattcttcgtattccctgtgtgcctattaccaagttatattgtttagtttgaggaaaacaacaaacaaccctcccttagagaatcatgatgcatattagcatatttaaggagtaccctaaagcttggacttaatctagcttaccctagacgtattaacatcctatagcgcacgtgttctgtgggatatatttcagaaaataatatgtcttacagaataaatgcagactcctttggatggcattcaggcccttcataaagtaactctgaattatttttcagtctcatttcctgcagttgcacccgattttttctgttctgcaatctgctattttagacaaagctttcataagaaattacataatgacaagtaattcccacattccctagatatatacttggatttcaaatgccataattgtaatctttctccaaagaaaaatgcacgatacaggcttcattctatcaagaggcatatgaggtaagcgttattatacacccgtccagcagatgggaaaaccacggaagtgggagggaatcaagaagcattaacagaccctccaccaaccgtaatgcttaactcctcttgttccaatcttaactcaaaattatgattattttgattatttaatgattgttttgatagtataaactccatatctgcaacctcagttatctacaacactgaggtgcaaacatttaaaaagatctctcaccaaacaaggattgaaattgtattgcaaatgaaagagctcagcatgagagcacaatcagaaactgaagagagttgatcaaagagaagattctagctgaattgttctatttttaattccccagggatcctacggtctatgacacattaacaggggtgtgaatcatttcagttacatcacatactctgtcctcttgtggtgcgggtcttccccagttgattatacacagataatcaatgactttatttgggagggattggagctatagaataatctatcttaccaatctgatggaccatttgtaagagctgtttgtcaaattggatcttcatttgacaaataaatactagagctgcagaaagtaaagtttactccaacccaattaatcattaatacatccacaggaaaaatacattttctccacatggaatattgatttttctatgaatattacctccacattgaaaaacgagtgagtcactgcagttaagccagtgtaccttgatgttgaattccctgaagaaggtacaattatatttacattcccttagcaagaatttggctaagtgataagcacttttctcttttcttttctttttttttttttttttttgagacagtgtctcactctgtcacccaggctggagggcagtagtgcagtctcagctcattgcaacctctgcctcccaggtttaagcaattctcgtgcctcagcctcctgaatagctgggattacaggaacatgccaccactcctggctaatttttgtatttttttttaagtaaagacaaactttcaccatgttggccaggctggtcttgaactcctgacctcttacgagctaatcataccacttctaactagattttctattaatgtacaaggagggactccactactaaagtttgttttacttctctggttatttacacaaaacgctggcaatacattaaggctcactaatgtggacctaagtgagtatctgagaggctttgaatatgtatgtgcaaactgtcctattttctttatatgctctcttaaatatgtatgtctgtaaatatatatataacacacatatatatatattcctagacatctagtgtttgctgtcattagtgaccaagaaaaagtagttcttttgtgcacgcgtgaatacatcaaattagcaattaccatagaaatgtatttcattgaataaatagcttttgtttgtttgtttgtttgtttcagggaaatttagaacaattattagatgttatagtgcctcttctcgtgttgatacgtgtatttgggtcaaaagtgcaaaaacttttttctacaatgtacagttattttgacttttcccaggggaagctagcaatagttttaaaagcacaaagtgatgtaaaatattttgacttttataataatgcgaagtagttttatttgcattaatcaataatgttcaggatctttttgtagtaagtatattttaaatggtgtaatttgtgtgtttatgatatgtattcaattatttaagttaagtatcagtgtatttttaaaaagtgttcccaggtgaacaaaagtttgcttttcttcaccattctgtacaggacaagcattattcagttcacatagtcagctctcactacacttcaacagaacggggcaagttcgtatttgaattctgttatatttatctaagggaagcaaaattaaccttttaataatagattatctttaatggttattaaaagaatgttattgaccaaaaagacatattttgagattaataaagatgtattagattatccaaaaaaaaaaaaaaaaa

SEQ ID NO: 2 - protein, 454 aa, Homo sapiens
MDHEAAQLEKQHVHNVYESTAPYFSDLQSKAWPRVRQFLQEQKPGSLIADIGCGTGKYLKVNSQVHTVGCDYCGPLVEIARNRGCEAMVCDNLNLPFRDEGFDAIISIGVIHHFSTKQRRIRAIKEMARVLVPGGQLMIYVWAMEQKNRHFEKQDVLVPWNRALCSQLFSESSQSGRKRQCGYPERGHPYHPPCSECSCSVCFKEQCGSKRSHSVGYEPAMARTCFANISKEGEEEYGFYSTLGKSFRSWFFSRSLDESTLRKQIERVRPLKNTEVWASSTVTVQPSRHSSLDFDHQEPFSTKGQSLDEEVFVESSSGKHLEWLRAPGTLKHLNGDHQGEMRRNGGGNFLDSTNTGVNCVDAGNIEDDNPSASKILRRISAVDSTDFNPDDTMSVEDPQTDVLDSTAFMRYYHVFREGELCSLLKENVSELRILSSGNDHGNWCIIAEKKRGCD

SEQ ID NO: 3 - DNA, 9313 bp, Homo sapiens
gtcttggttagttaaaaaaaaaaaaaaagttggccaggcacagtggcttgcacctgtaatcccagcactttgggaggccgaggcaggcggatcacctgaggttgggagttcgagaccaacctgaccaacatggagaaacccgtctctactaaaaatacaaaattatccggtcatggtggcacgcgcctatgatcccacctactcgggaggctaaggcaagagaattgcttgaacccgtgaggcagaggttgcagtgaaccgagattgcgccactgcactccagcctgggtgactgagggagactctgtctcaaaataaataaataaataaaaggttaattatatttcttgttatgagacaaatgaagtatttcacattttccattgaggatagcatgtaacgcaggtttttctcttataggttgtgggactggaaaatatcttaaagtgaacagccaggtacataccgtgggctgtgactactgtgggccactggtagagattgcccggaatagaggatgtgaagccatgtcatacatcatttttctacaaaacaaagaagaatcagagcaataaaagaaatggccagggtcttagttcccggaggccaactgatgatttacgtttgggcaatggaacaaaagaaccgtcactttgagaagcaagacgtgcttgttccatggaacagggctctgtgttcccagctcttctcagagtccagccagtctgggaggaagaggcagtgtggatacccagaaagaggccatccctaccatcctccttgctctgagtgtagctgttctgtttgttttaaagagcagtgtggttcaaaacggtcccacagcgtgggctatgaacctgctatggcaagaacctgttttgcaaatatttctaaggaaggcgaggaagaatatggattttacagcacattaggaaaatcgtttcgttcctggtttttctccagatctttggatgaatcgactctgaggaagcaaattgaaagagtaagacccttgaaaaacacagaagtttgggccagtagcactgtaacagtccagccttccagacactctagtttagactttgatcaccaagagccattttcaacaaaagggcaaagtttagatgaggaagtgtttgtggaatcttcttctggaaaacacttggagtggctgagagcaccaggcactctgaaacatttaaatggagaccatcaaggggaaatgaggagaaatggagggggaaattttctggatagcactaatactggtgtgaattgtgtggatgcaggcaacatagaagatgataatccttctgctagtaaaatattgagaaggatttctgcagttgattccacagatttcaacccagatgatacaatgtctgtcgaagatccacagactgatgttttggactccacagcctttatgcgctactaccatgtgtttcgagaaggggagctctgcagtctgctcaaggagaatgtgtcagagctccgtatcctgagttctgggaatgatcatggtaactggtgtatcattgcagagaaaaagagaggttgtgattgattggatccttttagacaactcctccaaaagatgaaccacattctttcctcttggtttgatatggttacctgaatttgcattcagtgttatttgttaatccatttacgctttggtctgcagagactattaattatttggttgttttgttttcatttttgaataagcacagattctggcattgaaagcacttgacaaagggtatttgtgcttaaatgttaatatacaagatctgaagaagcaacagaaagtacccttcagtacacctcagacttttttttaaccccagagagataaaatacatgtatagtgtttttcagtattacacattgatttaaaaagattatgctgttaaataatcttttaaaacggtatttttataaagtgaggggataatttctggttctcaggttataactgagagcagtgtgcaagataataggtaaatttgatccattgcacagatatactttgaaccatgtgatgagttatcttgttgccaaggccttgcttctacttaaagttttcagaaaactgagtgacagtggagagaaacaagaagttttacaaggactttactaaattataagcaaacttgcttcaaaataagttgacatgtgataataaggttttcaatgtagcccaggaggtttttaaaggcactgttaggctgagcatggtggctcatgcctgtaatcccagcactttgggaggccaaggcagaaggatcacttgaacccaggagttcaagaatagcctgggcaacatagcaagaccccatctctataaaaattagcaagatgtggtggtgcatgcctgtcatccaagctactcaagagactgaggcaggaggatcacgtgagcccaggaattcaaggctgcagtgaactatgattgcatcactgcactgcagcctgggcaacatagcaagactctgtctcaaaataataataataataataataaaggcgttgttagcttgtaaggagtggagtatgtaggtagtaggagttatatgcaagtacccaagtggtattcttccaatcttattagaagcatgaatattcaagattgatattactattgcttattagcaagattgttatcaatcatgcttattagaaggatgaatatccaagaccaagattgactaatgatgagtctgcatcaagaactaggcatttcttctgagttgacggactctttaggaaaggagaatctaagtgaagcactgattttagctctgagaacaaacaaattaaggtacagcatagttagccttggtagaggtatgacttggatttgctgtatcctttaaaatagtatctgggcatttattttattgaaggtgactacattttattagttatattaggaatttaggtagaatcaacttctactgattacaggttgaatttctgtcactttgtagagaaacgaatagactggacactgtgtggtcactgtttagatttgcccatgggtctctttaaatctatgtcatggatcctgagacacaaatataattaagacaggtctagagacaggagaagcagaaataagttgacccaggagtacagtctcaagtagttcattaatgagaaaattgacatctgacaagagtctttttactttatgctggatgaaaatccaaatcttgttttattttttccactaaaagtgactaaaataataacgaatttcatttgttcttgggttcttttttcctttaatgattgtgctataacttaaaataatgatgttacttttgaacaaacttaaagaaatatttttaaagcgtatctgaaaacgattgatgtttataactctcttttggcttcaaaataagattgtgttatcaccattttggtagatgaggttgtctggtgaaaatgatgcatatgagttgtactgttcagtgtacatcctgcagtagtggatgattgaaaacatatataagtggagtataaattaaaaattaatttggtttcttctatttcttttttttttttttttttttgagacagagtctcgctctgtcgcccaggctggagtgcactggcgcgatctcggctcactgtaagctccgcctcccgggttcacgccatcctcctgcctcagcctcccaagtagctgggactactggcacccaccacgacgcccggctgtttttttgtatttttaggagagacggggtttcaccgtgttagccaggatggtctcgatctcctgacgtcgtgatccacccgcctcggccccccacagtgctgggattacaggcgtgagccaccgcgcccggcctgttccttttatttcttaattcaggacactaaaccatgactgcaagggatttccttggtaaaaagaaaagattctcagagtcaaaatggtcttacaactcgggcttgacggcctttgaattatgaatggattgttcctctctctgaagcctattgtcacatgggtttttaatcctggccttgctgctagaaatctgtgcttgaagtcctctctttctgctgctagcctaccagttaaaagtcaagacttggtggaactcagtttaccagactctttagcctttgagctaaactgtctgagcaacctcttagatgtgcacacaccactttgtatgaaagggttctctagaacggttctttggagagaaatattttcatgtacgtttgacaggggtgtaaataaagcatgctgactaataagtcttttactcttcatctaatgaacataagaatctatgcatccagatattattttgtatacaaatatttaatttggtgattgataatctctctttggggtagtcacatggaaagctcttttaaatttaacttccgcctttggattttttttaaaaagccattgaagagcaaaactaatgtaaacgtcttgatcatttaaaaagcttgcttgtcctcgaaaggaaacacaggtcatcagtgagtataaacgtagacagttgatttgtgaatgctgtcggcctcaacttgcttgatgatagattctactgacctagctggagtaatctgatcacttacttccttattaatactagatcacacagtgtctttcttattccttcttctttacttactggcatcagcacagagtcccactatctgaaatagaagggagagtttgggggtttattggaggagtcttaaaaacttggttggcagctgggtacgatgactcacacctgtactcccagcacttcaggaagctgaggtgggtggatcagctgaggtcaggagtttgagaccagcctgaccaacatggtgaaaccccgtctttattaaatacaaaaaattagccaggttggtggcgcatgcctgtaatcccagctacttgggaggctgaggcaggagaatcgcttgaacctgggaggcggaggttgcagtgagccgagattgtgccattgcactccaacctgggcaacaagagtgaaactccatctccaacaaaacaaaactttgctggcttcctgatgcctcactgtctatttgaggaattccacagaatcttcaaaggatttgggggaaagcgcattaacatggacaaaggatggaatcaaaataatgttatagtgagaatcattcaagcacctatttaaattttttccaattgccagtatagtgatgatatgacaccagcatatcaaagtaactaacaaactagctacacaaacgtcttggagtttggtttcgttctcttttctcatcatagatctccgtgcagaatagtgctaattcttatttctcgtttgcctttctatttccttccaaattctacatgccagtaattcctctgtcttttaagtgaccatcaattcaataggcaaaaatttggagtaatccagagaaaaaaccatccaaatataaaccagctaggaacatgaatgcccctgattattaatggccaaaaaaaaagcactggtggattttaaattaaattaatacatatatacgagtttggaggagaacagaagttctaactcagtacttgaacttggtgggggagggcacaggttaaatatgagctgtgagcccccagttttggaggaaggaggaaatgggaaccaaccaccagacaagcagctgcagtctaagaaaaacaaattagggcaggcctcagcatctctttcctattacatagaaaatctgaaatgagaatagctgtgaacttttctaaatatgcaacattcaactatcgcaaggacaaaaaaccaaacaccgcatgttctcactcataggtgggaattgaacaatgagaacacatggacacagtaaggggaacatcacacactgggacctgttgtgaccccgctgagggagttcctcttgccctctacccccggggcttgtatatagattataaatatataagggggaaaggggtgggggggaggggttgtgcggctggggcctcacttcccctccttccccttctcctggtcccctgtcccaggggctgtttgttaaaaaagagtaataaaggatttttgtttttaaaaaaagcaacgttcattaatttatatgcagtcctcaccacagccctccaagataactatacttattgttcccattttgcagatgaggaaaactgaggcagaaagaaattaaattgcttaaggttacacacatagtaggtatcacacatttagttagaggcagagctgggattcaaacccaggtctggctcctagtctctgctcttaaccacaacaccatatactcccacccctgggcccagctcatgtgcttggcggagtattagagcctttcaaggtaagtgtgattatcccctttccaaatgagctttggaaaagtttaagcaaagattacacagctagacaataattcaatagtagtctcttacatattttcccttttatctttttttcatttgtaaatctgtttcgatttgatttgaattaaaaaatagttcttggttgcaaaatattctgggaccacttttctccaactcttcaacctttcaacaattatattattgttttcccattttataagtaaggcaactgaggactattcagatatttcattcactccatttgtttactaagccctgctgtgtgctggatacacagtctgctgtgtgctggatacatatgatacaaggtaatgactatctggcatcttgaaggtacccagaatattagaattaactgacccacaggtgccaagctgaaacattcttcgtattccctgtgtgcctattaccaagttatattgtttagtttgaggaaaacaacaaacaaccctcccttagagaatcatgatgcatattagcatatttaaggagtaccctaaagcttggacttaatctagcttaccctagacgtattaacatcctatagcgcacgtgttctgtgggatatatttcagaaaataatatgtcttacagaataaatgcagactcctttggatggcattcaggcccttcataaagtaactctgaattatttttcagtctcatttcctgcagttgcacccgattttttctgttctgcaatctgctattttagacaaagctttcataagaaattacataatgacaagtaattcccacattccctagatatatacttggatttcaaatgccataattgtaatctttctccaaagaaaaatgcacgatacaggcttcattctatcaagaggcatatgaggtaagcgttattatacacccgtccagcagatgggaaaaccacggaagtgggagggaatcaagaagcattaacagaccctccaccaaccgtaatgcttaactcctcttgttccaatcttaactcaaaattatgattattttgattatttaatgattgttttgatagtataaactccatatctgcaacctcagttatctacaacactgaggtgcaaacatttaaaaagatctctcaccaaacaaggattgaaattgtattgcaaatgaaagagctcagcatgagagcacaatcagaaactgaagagagttgatcaaagagaagattctagctgaattgttctatttttaattccccagggatcctacggtctatgacacattaacaggggtgtgaatcatttcagttacatcacatactctgtcctcttgtggtgcgggtcttccccagttgattatacacagataatcaatgactttatttgggagggattggagctatagaataatctatcttaccaatctgatggaccatttgtaagagctgtttgtcaaattggatcttcatttgacaaataaatactagagctgcagaaagtaaagtttactccaacccaattaatcattaatacatccacaggaaaaatacattttctccacatggaatattgatttttctatgaatattacctccacattgaaaaacgagtgagtcactgcagttaagccagtgtaccttgatgttgaattccctgaagaaggtacaattatatttacattcccttagcaagaatttggctaagtgataagcacttttctcttttcttttctttttttttttttttttttgagacagtgtctcactctgtcacccaggctggagggcagtagtgcagtctcagctcattgcaacctctgcctcccaggtttaagcaattctcgtgcctcagcctcctgaatagctgggattacaggaacatgccaccactcctggctaatttttgtatttttttttaagtaaagacaaactttcaccatgttggccaggctggtcttgaactcctgacctcttacgagctaatcataccacttctaactagattttctattaatgtacaaggagggactccactactaaagtttgttttacttctctggttatttacacaaaacgctggcaatacattaaggctcactaatgtggacctaagtgagtatctgagaggctttgaatatgtatgtgcaaactgtcctattttctttatatgctctcttaaatatgtatgtctgtaaatatatatataacacacatatatatatattcctagacatctagtgtttgctgtcattagtgaccaagaaaaagtagttcttttgtgcacgcgtgaatacatcaaattagcaattaccatagaaatgtatttcattgaataaatagcttttgtttgtttgtttgtttgtttcagggaaatttagaacaattattagatgttatagtgcctcttctcgtgttgatacgtgtatttgggtcaaaagtgcaaaaacttttttctacaatgtacagttattttgacttttcccaggggaagctagcaatagttttaaaagcacaaagtgatgtaaaatattttgacttttataataatgcgaagtagttttatttgcattaatcaataatgttcaggatctttttgtagtaagtatattttaaatggtgtaatttgtgtgtttatgatatgtattcaattatttaagttaagtatcagtgtatttttaaaaagtgttcccaggtgaacaaaagtttgcttttcttcaccattctgtacaggacaagcattattcagttcacatagtcagctctcactacacttcaacagaacggggcaagttcgtatttgaattctgttatatttatctaagggaagcaaaattaaccttttaataatagattatctttaatggttattaaaagaatgttattgaccaaaaagacatattttgagattaataaagatgtattagattatccaaaaaaaaaaaaaaaaa

SEQ ID NO: 4 - protein, 328 aa, Homo sapiens
MARVLVPGGQLMIYVWAMEQKNRHFEKQDVLVPWNRALCSQLFSESSQSGRKRQCGYPERGHPYHPPCSECSCSVCFKEQCGSKRSHSVGYEPAMARTCFANISKEGEEEYGFYSTLGKSFRSWFFSRSLDESTLRKQIERVRPLKNTEVWASSTVTVQPSRHSSLDFDHQEPFSTKGQSLDEEVFVESSSGKHLEWLRAPGTLKHLNGDHQGEMRRNGGGNFLDSTNTGVNCVDAGNIEDDNPSASKILRRISAVDSTDFNPDDTMSVEDPQTDVLDSTAFMRYYHVFREGELCSLLKENVSELRILSSGNDHGNWCIIAEKKRGCD

SEQ ID NO: 5 - DNA, 3884 bp, Pan troglodytes
aaattaagtgagaagagccgcgctaaagccaagcgtcgtcgtcacccaaggtcaaggggtggaagttacacctggtgcagccctcggctctgatgcaaaagcagcttttgcccctggctgcgggacagcgctgtgactactcgcatcgggagagctgctgccagtcgccacaccgtgcagaaagcgccggcgacccgagcactgacaatggtctgcataggggagcggagagaagcttctgttccgccctagatccgctgcctcggcgcccgcccgcagggaggagggggcgcgacaggtcgtctagcgcgtgccccggagcccgcgcccgggtctggccgcctggggcctgtgcttccttcagagactcacacaagtggtttatcatgagaaggaccgcactatttcatttcactcctacaagttttcatttacgttacacattgagaaagttatgagaagcaactgtcactctctggaggtggagactgccgtgattcacaaagacaagaggatggatcatgaagccgcccagctggagaagcagcatgtgcacaatgtgtacgagagcacagccccttacttcagcgacctgcagagcaaagcctggcctcgtgtccgccagttcctgcaagagcagaagccaggcagcctcatcgctgacataggttgtgggactggaaaatatcttaaagtgaacagccaggtacataccgtgggctgtgactactgtgggccactggtagagattgcccggaatagaggatgtgaagccatggtatgtgacaaccttaatctcccctttagggatgagggcttcgatgccatcatctccataggagtcatacatcatttttctacaaaacaaagaagaatcagagcaataaaagaaatggccagggtcttagttcctggaggccaactgatgatttacgtttgggcaatggaacaaaagaaccgtcgctttgagaagcaagacgtgcttgttccatggaacagggccctgtgttcccagctcttctcagagtccagccagtctgggaggaagaggcagtgtggatacccagaaagaggccatccctaccatcctccttgctctgagtgtagctgttctgtttgttttaaagagcagtgtggttcaaaacggtcccacagcgtggactatgaacctgctatggcaagaacctgttttgcaaatatttctaaggaaggcgaggaagaatatggattttacagcacattaggaaaatcgtttcgttcctggtttttctccagatctttggatgaatcgactctgaggaagcaaattgaaagagtaagacccttgaaaaacacagaagtttgggccaatagcactgtaacagtccagccttccagacattctagtctagactttgatcaccaagagccattttcaacaaaagagcaaagcttagatgaggaagtgtttgtggaatcttcttctggaacacacttggaatggctgagagcaccaggcactctgaaacatttaaatggagaccatcaaggggaaatgaggagaaatggagggggaaattttctggatagcactaataccggtgtgaattgtgtggatgcaggtaacttagaagatgataatccttctgctagtaaaatattgagaaggatttctgcagtcgattccacagatttcaactcagatgatacaatgtctgtcgaagatccacagactgatgttttggactacacagcctttatgcgctactaccatgtgtttcgagaaggggagctctgcagtctgctcaaggagaatgtgtcagagctccgtatcctgagttccgggaatgatcatggtaactggtgtatcattgcagagaaaaagggaagttgtgattgactggatccttttagacaactcctccaaaagatgaaccacattcttttctcttggtttgatatggttacctgaatttgcattcagtgttatttgttaatccatttacgctttggtctgcagagactattcattatttggttgtttttgttttcatgtttgaataagcacagattctggcattgaaagcacttgacaaagggtatttgtgcttaaatgttaatataaaagatctgaagaagcaacagaaagtgcccttcagtacagctcagacttttttttaaccccagagagataaaatacatgtatagtgtttttcagtattacacatcgatttaaaaagattatgctgttaaataatcttttaaaacggtatttttataaagtgaggggataatttctggttctcaggttataaatgagagcagtgtgcaagataataggtaaatttgatccattgcacagatatactttgaaccatgtgatgagttatcttgttgccaaggccttgcttctacttaaagttttcagaaaactgagtgacagtggagagaaccaagaagtgttacaaggactttactaaattataagcaaacttgcttcataataagttgacatgtgataataaggttttcaatgtagcccaggaggtttttaaaggcactgttaggctgagcatggtggctcatgcctgtaatcccagcactttgggagaccaaggcagaaggatcacttgaacccaggagttcaagaatagcctgggcaacatagcaagaccccatctctataaaaattagcaagatgtggtggtgcatgcctgtcatccaagctactcaagagactgaggcaggaggatcacgtgagcccaggaattcaaggctgcagtgaactatgattgcatcactgcactgcagcctgggcaacatagcaagactctgtctcaaaataataataataataataataataataataataaaggcattgttagcttgtaaggagtggagtatgtaggtagtaggagttatatgcaagtatccaagtggtattcttccaatcttattagaagcatgaatattcaagattgatattactattgcttattagcaagatttttatcaatcatgcttattagaaggatgaatatccaagaccaagattgactaatcatgagtctgcatcaagaactaggtatttcttctgagttgatggactctttaggaaaggagaatctaagtgaagcactgattttagctctgagaacaaacaaattaaggtacagcatagttagccttggtagaggtatgacttggatttgctgtatcctttaaaatagtatctgggcatttattttattgaaggtgactacattttattagttatattaagaatttaggtagaaacaacttctactgattaaaggttgaatttctgtcactttgtagagaaacgaatagactggacactgtgtggtcactgtttagatttgcccatgggtctgtttaaatctatgtcatggatcctgagacacaaatataattaagacaagtctagagacaggagaagcagaaacaagttgacccaggagtacagtctcaagtagttcattaatgagaaaattgacatctgacaagagtctttttactttatgctggatgaaaatccaaatcttgttttattttttccactaaaagtgactaaaataataacgaatttcatttgttcttgggttcttttttcctttaatgattgtgctataacttaaaataatgatgttacttttgaacaaacttaaagaaatatttttaaagcgtatctgaaaacgattgatgtttataactctcttttggcttcaaaataaggttgcgttatcacccttttggcagatgaggttgtctggtgaaaatgatgcatatgattgtactgttcagtgtacatcctgcagtagtggatgactgaaaacatatataagtggagtataaattaaaaattaatttggtttcttcta

SEQ ID NO: 6 - DNA, 3965 bp, Pan troglodytes
tggaagccccgacacgagccccggcccgcgcgcgcgctcccccacggccacgcgcgcaccctgccgcccgcacccccgcgcgccctccgtctatttttttctcttcctttcatcctcacattctaaaataggtcaaggggtggaagttacacctggtgcagccctcggctctgatgcaaaagcagcttttgcccctggctgcgggacagcgctgtgactactcgcatcgggagagctgctgccagtcgccacaccgtgcagaaagcgccggcgacccgagcactgacaatggtctgcataggggagcggagagaagcttctgttccgccctagatccgctgcctcggcgcccgcccgcagggaggagggggcgcgacaggtcgtctagcgcgtgccccggagcccgcgcccgggtctggccgcctggggcctgtgcttccttcagagactcacacaagtggtttatcatgagaaggaccgcactatttcatttcactcctacaagttttcatttacgttacacattgagaaagttatgagaagcaactgtcactctctggaggtggagactgccgtgattcacaaagacaagaggatggatcatgaagccgcccagctggagaagcagcatgtgcacaatgtgtacgagagcacagccccttacttcagcgacctgcagagcaaagcctggcctcgtgtccgccagttcctgcaagagcagaagccaggcagcctcatcgctgacataggttgtgggactggaaaatatcttaaagtgaacagccaggtacataccgtgggctgtgactactgtgggccactggtagagattgcccggaatagaggatgtgaagccatggtatgtgacaaccttaatctcccctttagggatgagggcttcgatgccatcatctccataggagtcatacatcatttttctacaaaacaaagaagaatcagagcaataaaagaaatggccagggtcttagttcctggaggccaactgatgatttacgtttgggcaatggaacaaaagaaccgtcgctttgagaagcaagacgtgcttgttccatggaacagggccctgtgttcccagctcttctcagagtccagccagtctgggaggaagaggcagtgtggatacccagaaagaggccatccctaccatcctccttgctctgagtgtagctgttctgtttgttttaaagagcagtgtggttcaaaacggtcccacagcgtggactatgaacctgctatggcaagaacctgttttgcaaatatttctaaggaaggcgaggaagaatatggattttacagcacattaggaaaatcgtttcgttcctggtttttctccagatctttggatgaatcgactctgaggaagcaaattgaaagagtaagacccttgaaaaacacagaagtttgggccaatagcactgtaacagtccagccttccagacattctagtctagactttgatcaccaagagccattttcaacaaaagagcaaagcttagatgaggaagtgtttgtggaatcttcttctggaacacacttggaatggctgagagcaccaggcactctgaaacatttaaatggagaccatcaaggggaaatgaggagaaatggagggggaaattttctggatagcactaataccggtgtgaattgtgtggatgcaggtaacttagaagatgataatccttctgctagtaaaatattgagaaggatttctgcagtcgattccacagatttcaactcagatgatacaatgtctgtcgaagatccacagactgatgttttggactacacagcctttatgcgctactaccatgtgtttcgagaaggggagctctgcagtctgctcaaggagaatgtgtcagagctccgtatcctgagttccgggaatgatcatggtaactggtgtatcattgcagagaaaaagggaagttgtgattgactggatccttttagacaactcctccaaaagatgaaccacattcttttctcttggtttgatatggttacctgaatttgcattcagtgttatttgttaatccatttacgctttggtctgcagagactattcattatttggttgtttttgttttcatgtttgaataagcacagattctggcattgaaagcacttgacaaagggtatttgtgcttaaatgttaatataaaagatctgaagaagcaacagaaagtgcccttcagtacagctcagacttttttttaaccccagagagataaaatacatgtatagtgtttttcagtattacacatcgatttaaaaagattatgctgttaaataatcttttaaaacggtatttttataaagtgaggggataatttctggttctcaggttataaatgagagcagtgtgcaagataataggtaaatttgatccattgcacagatatactttgaaccatgtgatgagttatcttgttgccaaggccttgcttctacttaaagttttcagaaaactgagtgacagtggagagaaccaagaagtgttacaaggactttactaaattataagcaaacttgcttcataataagttgacatgtgataataaggttttcaatgtagcccaggaggtttttaaaggcactgttaggctgagcatggtggctcatgcctgtaatcccagcactttgggagaccaaggcagaaggatcacttgaacccaggagttcaagaatagcctgggcaacatagcaagaccccatctctataaaaattagcaagatgtggtggtgcatgcctgtcatccaagctactcaagagactgaggcaggaggatcacgtgagcccaggaattcaaggctgcagtgaactatgattgcatcactgcactgcagcctgggcaacatagcaagactctgtctcaaaataataataataataataataataataataataaaggcattgttagcttgtaaggagtggagtatgtaggtagtaggagttatatgcaagtatccaagtggtattcttccaatcttattagaagcatgaatattcaagattgatattactattgcttattagcaagatttttatcaatcatgcttattagaaggatgaatatccaagaccaagattgactaatcatgagtctgcatcaagaactaggtatttcttctgagttgatggactctttaggaaaggagaatctaagtgaagcactgattttagctctgagaacaaacaaattaaggtacagcatagttagccttggtagaggtatgacttggatttgctgtatcctttaaaatagtatctgggcatttattttattgaaggtgactacattttattagttatattaagaatttaggtagaaacaacttctactgattaaaggttgaatttctgtcactttgtagagaaacgaatagactggacactgtgtggtcactgtttagatttgcccatgggtctgtttaaatctatgtcatggatcctgagacacaaatataattaagacaagtctagagacaggagaagcagaaacaagttgacccaggagtacagtctcaagtagttcattaatgagaaaattgacatctgacaagagtctttttactttatgctggatgaaaatccaaatcttgttttattttttccactaaaagtgactaaaataataacgaatttcatttgttcttgggttcttttttcctttaatgattgtgctataacttaaaataatgatgttacttttgaacaaacttaaagaaatatttttaaagcgtatctgaaaacgattgatgtttataactctcttttggcttcaaaataaggttgcgttatcacccttttggcagatgaggttgtctggtgaaaatgatgcatatgattgtactgttcagtgtacatcctgcagtagtggatgactgaaaacatatataagtggagtataaattaaaaattaatttggtttcttcta

SEQ ID NO: 7 - DNA, 7946 bp, Pan troglodytes
aactctgtcatcacctaggctggagtgcggtgatgctaccccagctcactgcaacctccacctcctgaatttaagtgattttttctgcctcagcttcctgagtaactgggaatacaggtacctgccaccacacccaggtaatttttgtatttttagtagagaggggtttcaccatgttggccaggctggtctcgaactcctgccctcaagtgatccgcccaccacggcctcccaaagtgccaggattataggcatgagccactgcaccagaccccaaattttattttttattcatatattctccactgtctcagctggcattaatgaatcttaagtttgcttttactgtatcataattttgaaagtgcctagtaaggatgggttattagtgtcctggtgtatttcagaagcagcaaactaatcatctttgaatagtggtagatatcttttcaggtagaaaaattttatcagtaatgccaataagtacaactataggctctaatgatttcttttaatatttctggaaagtaattagattatatatctgtgatagaatataatactaaatattagattgtagaaggctttgtttcaaggtggttattcattcatgttactaccttcctcagtcgtaacatcagtaagacctcaatgatctaaatctttgggctttgtaaatcctgaattacttaggtctctgctactggaaagtgaatatcaaaaattgtctatttaagaaaaaaagtagtttttataatctgagttgtatactggacccatagagatcattttaaatgtagcaatgaatagagattggataaaaataatgttaaaatttcttttctaattcaagttcaaattttaaaaatttgaaaggcatgttttagtgcataaataatagaaaagtcgttgacagttgtttgttttgcagtcagcaagtggtaagaatctttggagagacaacaggagaataaaggccgtaacatgcatatatacaaaatgaaatatgaatgtgagcttagaggctacacgtggtctctgggatactagaactatccttcttgccttggaccagctaacgtgccttggttgcgccagtgtactgagccctgtggacattacctcattaaatattcaataaatgttatttttttcctattgcctttctctcttctatatgagaatctgtaagtattcactgaaggcttttcttttgcttttgtgaataccttcacaccatgttgtattctgtcttggttagttaaaaaaaaaaaaaaagttggccaagtacagtggcttgcacctgtaatcccagcactttgggaggccgaggcaggcggatctcctgaggttgggagttcgagaccaacctgaccaacatggagaaacccatctctactaaaaatacaaaattatccggtcatggtggcacgcgcctatgatcccacctactcgggaggctaaggcaagagaattgcttgaacccgtgaggcagaggttgcagtgaactgagattgcaccactgcactccagcctgggtgactgagggagactctgtctcaaaataaataaataaataaaaggttaattatatttcttgttatgagacaaatgaactatttcacactttccattgaggatagcatgtaacgcaggtttttctcttataggttgtgggactggaaaatatcttaaagtgaacagccaggtacataccgtgggctgtgactactgtgggccactggtagagattgcccggaatagaggatgtgaagccatggtatgtgacaaccttaatctcccctttagggatgagggcttcgatgccatcatctccataggagtcatacatcatttttctacaaaacaaagaagaatcagagcaataaaagaaatggccagggtcttagttcctggaggccaactgatgatttacgtttgggcaatggaacaaaagaaccgtcgctttgagaagcaagacgtgcttgttccatggaacagggccctgtgttcccagctcttctcagagtccagccagtctgggaggaagaggcagtgtggatacccagaaagaggccatccctaccatcctccttgctctgagtgtagctgttctgtttgttttaaagagcagtgtggttcaaaacggtcccacagcgtggactatgaacctgctatggcaagaacctgttttgcaaatatttctaaggaaggcgaggaagaatatggattttacagcacattaggaaaatcgtttcgttcctggtttttctccagatctttggatgaatcgactctgaggaagcaaattgaaagagtaagacccttgaaaaacacagaagtttgggccaatagcactgtaacagtccagccttccagacattctagtctagactttgatcaccaagagccattttcaacaaaagagcaaagcttagatgaggaagtgtttgtggaatcttcttctggaacacacttggaatggctgagagcaccaggcactctgaaacatttaaatggagaccatcaaggggaaatgaggagaaatggagggggaaattttctggatagcactaataccggtgtgaattgtgtggatgcaggtaacttagaagatgataatccttctgctagtaaaatattgagaaggatttctgcagtcgattccacagatttcaactcagatgatacaatgtctgtcgaagatccacagactgatgttttggactacacagcctttatgcgctactaccatgtgtttcgagaaggggagctctgcagtctgctcaaggagaatgtgtcagagctccgtatcctgagttccgggaatgatcatggtaactggtgtatcattgcagagaaaaagggaagttgtgattgactggatccttttagacaactcctccaaaagatgaaccacattcttttctcttggtttgatatggttacctgaatttgcattcagtgttatttgttaatccatttacgctttggtctgcagagactattcattatttggttgtttttgttttcatgtttgaataagcacagattctggcattgaaagcacttgacaaagggtatttgtgcttaaatgttaatataaaagatctgaagaagcaacagaaagtgcccttcagtacagctcagacttttttttaaccccagagagataaaatacatgtatagtgtttttcagtattacacatcgatttaaaaagattatgctgttaaataatcttttaaaacggtatttttataaagtgaggggataatttctggttctcaggttataaatgagagcagtgtgcaagataataggtaaatttgatccattgcacagatatactttgaaccatgtgatgagttatcttgttgccaaggccttgcttctacttaaagttttcagaaaactgagtgacagtggagagaaccaagaagtgttacaaggactttactaaattataagcaaacttgcttcataataagttgacatgtgataataaggttttcaatgtagcccaggaggtttttaaaggcactgttaggctgagcatggtggctcatgcctgtaatcccagcactttgggagaccaaggcagaaggatcacttgaacccaggagttcaagaatagcctgggcaacatagcaagaccccatctctataaaaattagcaagatgtggtggtgcatgcctgtcatccaagctactcaagagactgaggcaggaggatcacgtgagcccaggaattcaaggctgcagtgaactatgattgcatcactgcactgcagcctgggcaacatagcaagactctgtctcaaaataataataataataataataataataataataaaggcattgttagcttgtaaggagtggagtatgtaggtagtaggagttatatgcaagtatccaagtggtattcttccaatcttattagaagcatgaatattcaagattgatattactattgcttattagcaagatttttatcaatcatgcttattagaaggatgaatatccaagaccaagattgactaatcatgagtctgcatcaagaactaggtatttcttctgagttgatggactctttaggaaaggagaatctaagtgaagcactgattttagctctgagaacaaacaaattaaggtacagcatagttagccttggtagaggtatgacttggatttgctgtatcctttaaaatagtatctgggcatttattttattgaaggtgactacattttattagttatattaagaatttaggtagaaacaacttctactgattaaaggttgaatttctgtcactttgtagagaaacgaatagactggacactgtgtggtcactgtttagatttgcccatgggtctgtttaaatctatgtcatggatcctgagacacaaatataattaagacaagtctagagacaggagaagcagaaacaagttgacccaggagtacagtctcaagtagttcattaatgagaaaattgacatctgacaagagtctttttactttatgctggatgaaaatccaaatcttgttttattttttccactaaaagtgactaaaataataacgaatttcatttgttcttgggttcttttttcctttaatgattgtgctataacttaaaataatgatgttacttttgaacaaacttaaagaaatatttttaaagcgtatctgaaaacgattgatgtttataactctcttttggcttcaaaataaggttgcgttatcacccttttggcagatgaggttgtctggtgaaaatgatgcatatgattgtactgttcagtgtacatcctgcagtagtggatgactgaaaacatatataagtggagtataaattaaaaattaatttggtttcttctatttcttttttttttttttttttttgagacagagtctccctctgtcgcccaggctggagtgcactggcgcgatctcggctcactgcaagctccccctcccgggttcacgccattctcctgcctcagcctcccgagtagctgggactacaggcacccaccacgacgcccggctgtttttttgtatttttagtagagacgggggtttcaccatgttagccaggatggtctcgatctcctgacgtcgtgatccacccgcctcggccccccaaagtgctgggattacaggcgtgagccaccgcccccggcctgtttcttttatttcttaattcaggacactaaaccatgactgcaagggattttcttggtaaaaagaaaagattctcagagtcaaaatgttcttacaactcgggcttgacggcctttgaattatgaatggattgttcctctctctgaagcctattgtcacatgggtttttaatcctggccttgctgctagaaatctgtgcttgaagtcctctctttctgctggtagcctaccacttaaaagtcaagacttggtggaactcagtttaccagactctttagcctttgagctaaactgtctgagcaacctcttagatgtacacacaccactttgtatgaaagggttctctagaacggttctttggagagaaatattttcatgtaagtttgacaggggtgtaaagaaagcatggcgactaataagtcttctactcttcatctaatgaacataagaatctatgcatccagatattattttgtatacaaatatttaacttggtgattgataatctctctttggggtagttacatggaaagctcttttaaatttaacttctgcctttggattttttttaaaaagccattgaagagcaaaactaatgtaaatgtcttgatcatttaaaaagcttgcttgtcctcgaaaggaaacacaagtcatcagtgagtataaacacagacagttgatttgtgaatgctgtcggcctcaacttgcttgatgatagattctactgacctagctggagtaatttgatcacttacttccttattaatactagatcacacagtgtctttcttattccttcttctttacttactggcatcagcacagagtcccactatctgaaatagaagggagagtttgggggtttattggaggagtcttaaaaacttggttggcagctgggtgcgatgactcacacctgtacccctagcacttcaggaggctgaggtgggtggatgaactgaggtcaggagtttgagaccagcctgaccaacatggtgaaaccccgtctttattaaatacaaaaaatttgccaggttggtggcgcatgcctgtaatcccagctacttgggaggctgaggcaggagaatcgcttgaacctgggaggcggaggttgcagtgagccaagattgtgccattgcactccaacctgggcaacaagagtgaaactccatctcaaacaaaacttttttggcttcctgatgcctcactgtctatttgaggaattccacggaatcttcaaaggatttgggggaaagcgcattaacatggacaaaggatggaatcaaaataatgttatagtgagaatcattcaagcacctatttaaattttttccaattgccagtatactgatgatatgacaccagcatatcaaagtaactaacaaactagctacacaaacgtcttggagtttggtttggttctcttttctcatcacacatctccgtgcagaatagtgctaattcttatttctcgtttgcctttctatttccttccaaattctacatgccagtaattcctctgtcttttaagtgaccatcaattcaataggcaaaaatttggagtaatccagagaaaaaaccatccaaatataaaccagctaggaacatgaatgcccctgaatattaatggccaaaaaaaagcactggtggattttaaattaaattaatacatatatacgagtttagaggagaacagaagttctaactcagtagttgaacttggtgggggagggcacaggttaaatatgagctgtgagcccccagttttggaggaaggaggaaatgggaaccaaccaccagccaagcagctgcagtataagaaaaacaaattagggcaggcctcagcatctctttcctattacatagaaaatctgaaatgagaatagctgtgaacttttctaaatatgcaacattcaactatcgcaaggacaaaaaaccaaacaccgcatgttctcactcataggtgggaattgaacaatgagaacacatggacacaggaaggggaacatcacacactggggcctgttgtgaccccgctgagggagttcctcttgccctctacccccggggcttgtatatagattataaatatataagggggaaaggggtggggggtaggggttgtggggctggggcctcacttcccctccttccccttcccctggtcccctgtcccaggggctgtttgttaaaaaagaggaataaaggaatttttttttttttaaaaaagcaacgttcattaatttatatgcagtcctcaccacagccctccaagataagtatacttattgttcccattttgcagatgaggaaaactgaggcagaaagaaattaaattgcttaaggttacacacatagtaggcatcacacatttagttagaggcagagctgggattcaaacccaggtctggctcctagtctctgctcttaaccacaacaccatatactcccacccctggacccagctcatgtgcttggcagagtattagagcctttcaaggtaagtgtgattatcccctttccaaatgagctttggaaaagtttaagcaaagattacacagctagacaataattcaatagtagtctcttacatattttcccttttatcttttttcatttgtaaatctgtttcgatttgatttgaattaaaaaatagttcttggttgcaaaatattctgggaccacttttctccaactcttcaacctttcaacaattattgttttcccattttataagtaaggcaactgaggact

SEQ ID NO: 8 - DNA, 1773 bp, Canis familiaris
atggaccatgaagccgcccagctggagcagcagcatgtgcatgatgtgtatgagagcaccgcaccatacttcagcgacctgcagagcaaagcatggcctcgcgtccgccagttcctccaggagcagaagccgggcagcctcattgctgacataggttgtgggactggaaagtatcttaaagtgaacagccaggtgtatactctgggctgtgactactgtgggccattggtagagattgcccggagtagaggatgtgaagtcatggtatgtgacaaccttaatctcccctttagggatcagggcttcgatgccatcatctccataggagtcatacatcatttttctacaaaacagagaagaatcagagcaattaaagaaatggccagggtcttagttcctggaggccagctaatgatttacgtgtgggccatggaacaaaagaaccgacattttgagaaacaagatgtgctcgttccatggaacagggctttatgctcccagcttttctcagactccagccagcctgggagaaagcagcagtgtggacatccagaaagaagccattcctaccaccctccttgctccatctgtagctgttctgtttgttttaaagagcagtgtccttcaaaacggtcccacagcatggactatgaaccactgatggctggaaactgttgtgcagatgtttctaaggaaggggaggaagaaaatggattctataacaccttaggaaaatcttttcgttcctggtttttctctaggtctttggatgaatcaactctgaggaagcagattgaaagagcaagacccttgaaaaatacagaaggttgggccaatagcacaatatccatccagccttccagacactccagcctagacttagatcatcaagagccattttcaataagagaacaaaatttagacgaagatgtgtttgtggagacttctcagaaaccactggagtggccaagagcatcagccgcagtgaaacatctacatggagaccaacaaggagaagttaggagaaatggagatgggaattttctggaaggcgcccatcccagtgacaattgtgtgaatgccggtaatgtggaagatgggaacccttctgctagtaaaatactgaaaaggatttctgcactcaactccacagattccaacccagatgagacaatttctgtcaaagaacaacagcccaacgttttggattccagagcctttatgcgctactaccatgtgtttcgagaaggtgagctctatggcctgctgaaggagcacgtggcagagctgcatgttctgagctctgggaatgatcatggcaactggtgtatcattgctgaaaaaaaggacaattgggactgagtggatcattttagacaattcctccaaaaggtgaaccatgttctttctttactaagtttggtatggttacctggatttgcatacaattttactatttttaatccatttatgctttggttcatggagatgatgaattaatcatcttttaaaccttcagacgaacatagagcctgtaaatgtaaaacaaatgtaaaacatttgacaaaggatgttcataagttatctatagagaactgaagaagcaataggaaatgaacttcagtatggttgggatcttttcttccctgaaaggtgaaattattttgaggacacacgtatatcatttttcagtattacaggcagattttaatagattctggttgttgacttttttttattactattgaagtagagtcaacagatgatatcata

SEQ ID NO: 9 - DNA, 1825 bp, Mus musculus
gccaccatgattgaccaaaacaagaagtaatccacgtgatcacaagatgagtcaggatggatcctgaagctgtggaactggagaagcgacatgtgcacagtgtgtacgagaacactgccccctacttcactgacctgcagagcaaagcctggccgcgtgtccgccagttcctgcaggaccagaagccaggcagcctcgtcgctgacattggttgtggaactggaaagtatcttaaagtgaatagccaagtccatactctgggctgtgactactgtgggcctctggtagagattgcccggaatagagggtgtgaagtcatggtatgtgacaaccttaatcttccctttagggaccagggcttcgatgctatcatctccataggagtcatacatcatttttctacaaaagaaagaagaattagagcaataaaggaaatggccagggttttagctcccggaggccagttgatgatttatgtctgggcaatggaacagaagaaccgacgctttgagaaacaggatgtacttgtgccgtggaacagggcactctgttcccggctgctgtcagaatcccaccagtcatggggtcatcactgtgagcatcccaggagccgaggcttccagggccccggttccgtgtgcggctgcgcagtgtgttttaagggcagatgtgattccaaacggtcccacagcatggactatgggtctgcggtggccagaacctgctgcgaagctatttcaaaggaaggcgagcgagagaacggattgtatagcaattttgggaaatcctttcgttcctggtttttctccaggtctttggatgaatcaaccctgaggaagcaaattgaaagagtcaggcccatgaaaatcccagaggcttgggccaatagcaccgtgtcccaacagccttccagacaccccagcttagacttgcacgctccggagccattttcaacaaaaggaccaaacttagatgaggtgttcgtagacacatcttctcaaagacacttgggctggctgagaacacctggcacatcagacaacttcagtggacacaagggaggagggagcaggaggaaggaggggggcaattttctggacatcactgataccggggacagtgtggctgctagtaacagcagtgatccttctgccagaaaaatattaaggagggtttctgcattcgactccaacgactccaactcagaagactcaagctttttggaagcacaacgggatgccaccgattccaaagccttcatgcgttactaccatgtatttcgggaaggtgagctctccagcctgctgcaggagagtgtgtctgagctacaggttctgagctccggcaatgaccatggtaactggtgcatcattgcagagaaaaagaggagctgggattaaacgacacggctcaaggcagaggctacggtggtggcctatcatctgttcaacggggctggtgtgggtaccagaagcagcatcctgtttcattgttttcaaatccaggtacgagttggtacttaagcactttgaccgcatcttcctaattgttgagtaagcacgaagtctgacacgtactgcatctgctacaggatctgcctgctgaagtttcaataagagaatctgaggaagcaataggaactgaactttaagtataggcttgatcttcttcctcccagtgatgaaattattataatatgtgtatgtcaagcgtcagtattatcttttggtttttaaatattctgctgttaagtataccctttaaaagcagtttgtttgtttgtttgtttgtttgttttaaataaattgcattggttctcaggtaa

SEQ ID NO: 10 - DNA, 2491 bp, Rattus norvegicus
cagatacccggtagagatttttcctttacagtgtgtgttgtggtcaggatcaaaagggaagagttaggcttagggtcacgtgggtgtcaccacaggtactgagctgcgcttggggccccgccaggtgtacccgcccagaggcgtctcccgcttaggacccttcaccccaccggacactggagtagagcgaggtgagagacaggaaggtcatcctgattccagcctcaggcacgtccgggcgcggcgatcgcaggggacagacagagcggccacaccgcttgggctctgccgcgcgcccgcgccgcaccgagccgactccgaggcgccccgggcttgcggccgacccatggaagttgcatcctggacagtccgggctgcgacgctgaagagcaccgagccagtgctcggccgagcctcccgcccggatcccgggcaaagaccgcccaaaacctgctgttgaccttcgtctgcactgctcaatggtgacccgagtcttcgctgtcctagttgcagccatttggcatccccaagactgccagagcacccgggatctgcaaacctggggcttggattctcttcagagactcatactgagtggtggatcttgtgaagggctcactaactcaattcctgctcctttcatgtgtgttatatactgagagccctgagaaggaactgttcctccctgaggaaggaaactgccgtgatttaccaaaaccagaaataatccacatgatcacaaaatgagtcaggatggatcccgaagccgtgcagctggagaagcagcacgtgcacaaggtctatgagagcaccgccccctacttcagtgaccttcaaaacaaagcttggccccgcgtccgccagttcctgcaggaccagaagccaggcagcctcatcgctgacattggttgtggaactggaaagtatcttaaagtgaatagccaagtacatactctgggctgtgactactgtggccctctggtagagattgcccggaatagagggtgtgaagtcatggtatgtgacaaccttaatctcccctttagggaccagggcttcgatgctatcatctccataggagtcatacatcatttctctacaaaacagagaagaatcagagcaataaaggaaatggccagggtcttagctcccggaggccagctgatgatttacgtctgggcaatggaacagaagaaccgacactttgagaaacaggatgtgcttgtgccatggaacagggcactctgttcccggctgctgtcagaatcccaccagtcgtgggggcattcgtgtgagcatcccacgagccaaggctaccagggccctggctccgcgtgcagctgtgcagtgtgttttaagggcagatgtgattccaagcggtcgcacagcatggactacgggcctgcggtggccagaacctgctgcgaagctatttcgaaggaaggacagcaagagaatggattatatagcaattttggaaaatcctttcgctcctggtttttttccaggtctttggatgaatcaaccctgaggaagcaaattgaaagggtcaggccgatgaaaatcgagggctgggccaacagcaccgtgtcccaccagccttcccgataccccagcgtagacttgcatgcccaggagccgttttcaacaaaaagaccgaacttagatgaggtgttcgtagacgcatcttctcaaagacacgtgggctgcctgggagtacccagcacttcagacaacttcattggacacaagggaggagagagtaggaggaaggaggggggcaattttctggacaccactgataccgggaacagtgtggctgcaagaaatggcggtgatcgttctgccagaaaaatattaaggagggtttctgcattcgactccacggactccacctcagaagacccaagctttgtggaagggcaacaggatggcccagattccaaagccttcatgcgttactaccacgtgttccgggaaggtgagctgtccagcctgctgcaggagtgtgtgtctgagctgcaggttctgagctccggcaatgaccatggtaactggtgcatcatcgcagagaaaaagaggagctgggattaactacatcgctcaaggcagaggctacagcccatcatctgctcaatggggctgatgtggacaccagaatcggcaccctagttcattgctttcaaatccaggtatgagtcggaccgtgagcactgtgccctcatcctccgaactgttgagtaagcatgaaggctggcatgtcctgcctctgctgcgggatctgcctgctgaagtttcaatcaaaggatccgaggaagcagtagaaagtaaacttaagtacagacttgatcttattcctcccagagatgaaattattacgatacgtgtacgtcaagtgtcagtattatcttttggtttttaaataatcccctgttaaatataccttttaaaagtagtaatttttaaaaataaattatgttggttctcaggt

(19) United States
(12) Patent Application Publication
(10) Pub. No.: US 2014/0329701 A1
(43) Pub. Date: Nov. 6, 2014

(54) KIAA1456 EXPRESSION PREDICTS SURVIVAL IN PATIENTS WITH COLON CANCER

(71) Applicant: Fátima Valdés Mora, Rushcutters Bay (AU)

(72) Inventors: Juan Carlos Lacal SanJuan, Madrid (ES); Fátima Valdés Mora, Rushcutters Bay (AU)

(73) Assignee: TRASLATIONAL CANCER DRUGS PHARMA, S.L., Valladolid (ES)

(21) Appl. No.: 14/117,037

(22) PCT Filed: May 11, 2012
(86) PCT No.: PCT/EP2012/058778

§ 371 Date: Jun. 23, 2014

Related U.S. Application Data
(60) Provisional application No. 61/490,658, filed on May 27, 2011.

(30) Foreign Application Priority Data
May 12, 2011 (EP) 11382144.1

(51) Int. Cl. C12Q 1/68

(52) U.S. Cl. CPC C12Q 1/6886; C12Q 2600/106; C12Q 2600/158; C12Q 2600/118
USPC 506/9; 435/6.11; 435/6.12; 435/7.4; 506/16

(57) ABSTRACT

The invention relates to methods for deciding on the therapy in a subject suffering from colorectal cancer as well as for predicting the clinical outcome of a patient which suffers from colorectal cancer based on the expression level of KIAA1456 comprising determining the expression level of the KIAA1456 gene. The invention relates as well to kits and uses thereof comprising reagents adequate for the determination of the expression level of the KIAA1456 gene.

### FIELD OF THE INVENTION

The invention relates to the field of diagnostics, in particular to a method of providing personalized diagnosis to colon cancer patients based on the expression of certain biomarkers in a sample from said patients.

### BACKGROUND OF THE INVENTION

Colorectal cancer, also called colon cancer or large bowel cancer includes cancerous growths in the colon, rectum and appendix. With 655,000 deaths worldwide per year, it is the third most common form of cancer and the second leading cause of cancer-related death in the Western world. Many colorectal cancers are thought to arise from adenomatous polyps in the colon. These mushroom-shaped growths are usually benign, but some may develop into cancer over time. The majority of the time, the diagnosis of localized colon cancer is through colonoscopy.

Surgical intervention is the main mode of treatment for patients in early stages (from stage I to III). However, as the tumor stage increases in terms of depth of penetration of the tumor in the intestinal layers and involvement of lymph nodes, the possibility of a cure by surgery alone decreases and adjuvant chemotherapy and radiotherapy is required.

The following stand out among the studied molecular factors which seem to be clinically relevant: the levels of the carcinoembryonic antigen (CEA) and the carbohydrate antigen CA19-9 in serum [Yamashita K, Watanabe M. Cancer Science 2009; 100:195-199], the loss of heterozygosity of chromosome 18q and high microsatellite instability (MSI) [Compton C C. et al; College of American Pathologists Consensus Statement 1999. Archives of Pathology & Laboratory Medicine 2000; 124:979-994].

A recent work by Yamashita et al. reviews the clinical relevance of emerging tumor markers in CRC [Yamashita K, Watanabe M. Cancer Science 2009; 100:195-199]. The authors summarize the possible prognostic markers according to the tumor stage, since the patient's survival generally depends on these stages.

The detection of prognostic markers is crucial in stage II, especially for improving the selection of patients who must receive adjuvant chemotherapy after surgery to prevent recurrence as well as the patients who will be sensitive to said treatment. In this sense, it has been found that aneuploidy, specifically the specific count of the 8p and 18q alleles, can predict recurrence in stage II patients [Zhou W. et al; Lancet 2002; 359: 219-225]. Furthermore, a profile of 23 altered genes in Dukes B patients which predict recurrence has also been demonstrated by means of a transcriptome analysis by cDNA microarrays [Wang Y. et al; J. Clin. Oncol., 2004; 22: 1564-1571]. However, a consensus has not been reached on these results due to the variability of the array platforms used. Therefore, none of the gene expression markers identified has been implemented for a clinical application.

Prognostic markers for stage III have still not been identified either. The detection thereof would be very important, especially when selecting patients who must receive the most potent and expensive chemotherapy treatments, as well as for the development of new therapeutic targets for the treatment of patients with recurrence and resistance to conventional therapies. In this sense, it has been demonstrated that the K-ras mutation in primary tumors indicates a worse prognosis in the patients of this stage [Andreyev H J. et al; British Journal of Cancer 2001; 85: 692-696]. Furthermore, it has been found that this mutation must be used to predict the therapeutic effect of the epidermal growth factor receptor (EGFR) inhibitor cetuximab or panitumumab, since patients with CRC affected by the K-ras mutation are insensitive to the treatment by EGFR inhibition [Benvenuti S. et al; Cancer Research 2007; 67: 2643-2648]. Again, at present no prognostic marker for stage III CRC has yet been applied to clinical practice.

In stage IV patients it has been detected that high levels of preCA19-9 (preoperative levels of CA19-9) are a biomarker of worse prognosis. There are no molecular prognostic or response factors consolidated for their clinical application to date.

The presence of tumor cells in regional lymph nodes in stage III patients predicts 60% recurrence at 5 years. The treatment with chemotherapy of these patients after surgical intervention reduces the recurrence by 40% to 50% and is currently the standard care given to stage III patients.

Stage I and II patients do not have affected lymph nodes or distal metastasis and therefore they have a better prognosis. Surgical intervention is highly effective when the disease is localized; however a proportion of 25-30% of stage II patients experiences recurrence and dies due to this disease. The benefit of postoperative chemotherapy is not clear for this latter group of patients. Various studies have demonstrated that there are no differences in survival between stage II patients treated with adjuvant chemotherapy and those who have not received it [J. Clin. Oncol. 1999; 17:1356-1363]. In contrast, a review of the National Surgical Adjuvant Breast and Bowel Project sets forth that adjuvant chemotherapy does improve survival in certain stage II patients [Mamounas E. et al; J Clin Oncol 1999; 17:1349-1355].

The detection of prognostic markers in stage II patients is critical when determining patients who are predisposed to experiencing recurrence and who will be sensitive to the treatment with chemotherapy. The reduction of the number of patients who experience side effects from chemotherapy without obtaining any therapeutic effect would also be achieved with this selection. Therefore there is still a need for further markers useful for predicting the clinical outcome of colon cancer patients and stage II patients.

### SUMMARY OF THE INVENTION

In a first aspect, the invention relates to a method for deciding on the therapy in a subject suffering from colorectal cancer comprising determining the expression level of the KIAA1456 gene in a sample from said subject, wherein altered expression level of said gene when compared to a reference level is indicative that said therapy is adequate for said patient.

In another aspect, the invention relates to a method for predicting the clinical outcome of a patient suffering from colorectal cancer comprising determining the expression level of the KIAA1456 gene in a sample from said patient wherein an increased expression level of the KIAA1456 gene in said sample when compared with a reference level is indicative of a poor clinical outcome or wherein the same or a decreased expression level of the KIAA1456 gene in said sample when compared with a reference level is indicative of a good clinical outcome.

In another aspect, the invention relates to a kit comprising a set of reagents capable of specifically detecting the expression level of KIAA1456 and, optionally, a housekeeping gene or the protein encoded by said housekeeping gene.

In yet another aspect, the invention relates to the use of the kit according to the invention for determining the need for treatment with a therapeutic agent or a combination of therapeutic agents of a subject or for predicting the clinical outcome of a subject suffering from colorectal cancer.

### BRIEF DESCRIPTION OF THE FIGURES

FIG. 1. Gene expression levels of KIAA1456 gene isoform 1 in 80 samples of patients with stage II CRC. A) RQ value of each tumor sample (grey bars) and of the control normal tissue (indicated by the black line and bar). B) KIAA1456 gene expression pattern calculated by the RQ method and shown as Log₁₀ RQ, where the 0 value is the expression in the normal tissue, expression values less than zero indicate a silencing whereas positive values indicate overexpression with respect to the normal tissue used as a reference.

FIG. 2. Correlation between KIAA1456 expression and clinicopathological parameters. A) Mean comparison between the RQ value of KIAA1456 in the tumor samples and perineural invasion (p=0.004, Mann-Whitney test). B) Mean comparison between the RQ value of KIAA1456 in the tumor samples and intestinal obstruction/perforation at the time of diagnosis (p=0.003, Mann-Whitney test).

FIG. 3. Survival according to transcriptional KIAA1456 gene overexpression in patients with stage II CRC. Kaplan-Meier curve in which the patients are grouped according to KIAA1456 expression and the disease-free survival is plotted over the follow-up time (p=0.05). Black solid line indicates low KIAA1456 expression and grey dashed line indicates high KIAA1456 expression.

FIG. 4. Survival according to the cases of no vascular or perineural invasion. Kaplan-Meier analysis was performed stratifying patients regarding the presence or absence of vascular or perineural invasion. A), B) Kaplan-Meier curves for relapse-free survival of the patients when AQ values of KIAA1456 expression were correlated with the outcome of the patients. C), D) Kaplan-Meier curves for relapse-free survival of the patients when RQ values of KIAA1456 expression were correlated with the outcome of the patients.

FIG. 5. Survival according to significant KIAA1456 overexpression stratifying according to whether or not the patients have received adjuvant chemotherapy. A) Analysis of the overall survival of the patients according to significant KIAA1456 overexpression depending on whether the patients did not receive chemotherapy (p=0.01) or did receive it (B). Analysis of the disease-free survival of the patients according to significant KIAA1456 overexpression depending on whether the patients received chemotherapy (D) or did not receive it (C) (p=0.012). Black solid line indicates low levels of KIAA1455 and grey dashed line indicates high levels of KIAA1455.

FIG. 6. Survival according to greater KIAA1456 overexpression stratifying according to whether or not the patients received adjuvant chemotherapy. A) Kaplan-Meier curve of the overall survival over the follow-up time in which the patients are grouped according to KIAA1456 overexpression (establishing an arbitrary cut-off point of 69^{th} percentile) and depending on whether the patients did not receive chemotherapy (p=0.031) or did receive it (B) (non-significant). C) Kaplan-Meier curve of the disease-free survival over the follow-up time in which the patients are grouped according to KIAA1456 overexpression (establishing an arbitrary cut-off point of 64^{th} percentile) depending on whether the patients did not receive chemotherapy (p=0.025) or did receive it (D) (non-significant). Black solid line indicates levels of KIAA1456 expression below the cut-off point and grey dashed line indicates KIAA1456 expression levels over the cut-off point.

### DETAILED DESCRIPTION OF THE INVENTION

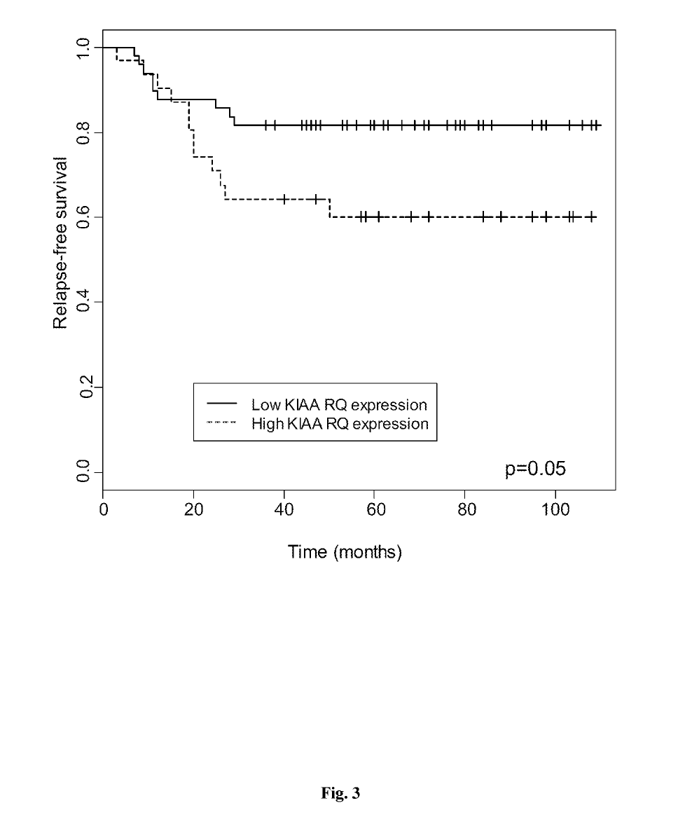

The authors of the present invention have found that the clinical outcome of patients suffering colon cancer closely correlates with the expression level of KIAA1456 (see FIGS. 3 to 6) and that increased expression level of KIAA1456 is a marker for poor clinical outcome of the patient (see FIGS. 3 to 6). These findings allow deciding whether a patient suffering from cancer is at risk of poor outcome and thus, it is a candidate to be treated with a given therapy.

### Method for Deciding on the Therapy in a Subject

In a first aspect, the invention relates to a method (hereinafter first method of the invention) for deciding on the therapy in a subject suffering from colorectal cancer comprising determining the expression level of the KIAA1456 gene in a sample from said subject, wherein altered expression level of said gene when compared to a reference level are indicative that said therapy is adequate for said patient or wherein the same expression level of the KIAA1456 gene in said sample when compared to a reference level is indicative that said therapy is not adequate for said patient.

As used herein, the term “deciding” means carrying out an assessment on the convenience of treating a patient with a given therapy. The skilled person will understand that patients which are predicted to have an unfavourable clinical outcome will be candidates for receiving a therapy, whereas patients having a low risk of having an unfavourable clinical outcome can be spared the undesired effects of the therapy. As will be understood by those skilled in the art, such an assessment is usually not intended to be correct for all (i.e. 100 percent) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley and Sons, New York 1983. Preferred confidence intervals are at least 90 percent, at least 95 percent, at least 97 percent, at least 98 percent or at least 99 percent. The p-values are, preferably, 0.1, 0.05, 0.01, 0.005, or 0.0001. More preferably, at least 60 percent, at least 70 percent, at least 80 percent or at least 90 percent of the subjects of a population can be properly identified by the method of the present invention.

As used herein, the term “therapy”, when referred to colorectal cancer, refers to any attempted remediation or prevention of the appearance of colorectal cancer or of a metastasis thereof and includes, without limitation, radiation therapy, chemotherapy, immunotherapy and combinations thereof.

The term “radiation therapy” is usually defined as the use of high-energy radiation from x-rays, gamma rays, neutrons, protons, and other sources to kill cancer cells and shrink tumors.

The term “chemotherapy” as used herein refers to the treatment of cancer using specific chemical agents or drugs that are destructive of malignant cells and tissues. In the particular case of colorectal cancer, chemical agents that are commonly used include, without limitation, platinum drugs, pyrimidine antimetabolite drugs, leucovorin and combinations thereof.

The term “immunotherapy” refers to an array of treatment strategies based upon the concept of modulating the immune system to achieve a prophylactic and/or therapeutic goal. In a particular embodiment, the immunotherapy involves the use of anti-VEGF antibodies and, most preferably, bevacizumab.

“Platinum drugs” refer to any anticancer compound that includes platinum. In an embodiment, the anticancer drug can be selected from cisplatin (cDDP or cis-iamminedichloroplatinum(II)), carboplatin, oxaliplatin, and combinations thereof. “Oxaliplatin” (Eloxatin®) is a platinum-based chemotherapy drug in the same family as cisplatin and carboplatin. Equivalents to Oxaliplatin are known in the art and include, but are not limited to cisplatin, carboplatin, aroplatin, lobaplatin, nedaplatin, and JM-216.

Pyriminidine antimetabolite drug or therapy includes, without limitation fluorouracil (5-FU), which belongs to the family of therapy drugs call pyrimidine based anti-metabolites. 5-FU is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-I (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487. For the purpose of this invention, pyrimidine antimetabolite drugs include 5-FU based adjuvant therapy.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid ?[4[[(2-amino-5-formyll,4,5,6,7,8hexahydro4oxo6-pteridinyl)methyl]amino]benzoyl], calcium salt (1:1).

“FOLFOX” is an abbreviation for a type of combination therapy that is used to treat cancer. This therapy includes 5-FU, oxaliplatin and leucovorin. “FOLFIRI” is an abbreviation for a type of combination therapy that is used treat cancer and comprises, or alternatively consists essentially of, or yet further consists of 5-FU, leucovorin, and irinotecan. Information regarding these treatments is available on the National Cancer Institute's web site, cancer.gov, last accessed on Jan. 16, 2008. Equivalents of

FOLFOX/BV intend where one or more of the components of the composition are substituted with an equivalent, e.g., an equivalent to 5-FU and/or oxaliplatin.

“XELOX/BV” is another combination therapy used to treat colorectal cancer, which includes the prodrug of 5-FU, known as Capecitabine (Xeloda) in combination with oxaliplatin and bevacizumab. Equivalents of XELOX/BV intend where one or more of the components of the composition are substituted with an equivalent, e.g., an equivalent to bevacizumab and/or oxaliplatin. Information regarding these treatments is available on the National Cancer Institute's web site, cancer.gov or from the National Comprehensive Cancer Networks web site, nccn.org, last accessed on May 27, 2008.

The terms “colon cancer” or “colorectal cancer” relates to a tumour of the colon and includes any histology subtype which typically appears in colon cancer such as transitional cell carcinomas, squamous cell carcinoma and adenocarcinoma, any clinical subtype such as superficial, muscle-invasive or metastatic disease cancer and any stage.

Colon cancer staging is an estimate of the amount of penetration of a particular cancer. It is performed for diagnostic and research purposes, and to determine the best method of treatment. The systems for staging colorectal cancers depend on the extent of local invasion, the degree of lymph node involvement and whether there is distant metastasis.

The most common staging system is the TNM (for tumors/nodes/metastases) system, from the American Joint Committee on Cancer (AJCC). The TNM system assigns a number based on three categories. “T” denotes the degree of invasion of the intestinal wall, “N” the degree of lymphatic node involvement, and “M” the degree of metastasis. The broader stage of a cancer is usually quoted as a number I, II, III, IV derived from the TNM value grouped by prognosis; a higher number indicates a more advanced cancer and likely a worse outcome. Details of this system are in Table 1.

TABLE 1
TNM system for the staging of colorectal cancer

| AJCC | | TNM stage criteria for colorectal |
|---|---|---|
| stage | TNM stage | cancer |
| Stage 0 | Tis N0 M0 | Tis: Tumor confined to mucosa; |
| | | cancer-in-situ |
| Stage I | T1 N0 M0 | T1: Tumor invades submucosa |
| Stage I | T2 N0 M0 | T2: Tumor invades muscularis propria |
| Stage II-A | T3 N0 M0 | T3: Tumor invades subserosa or beyond |
| | | (without other organs involved) |
| Stage II-B | T4 N0 M0 | T4: Tumor invades adjacent organs or |
| | | perforates the visceral peritoneum |
| Stage III-A | T1-2 N1 M0 | N1: Metastasis to 1 to 3 regional |
| | | lymph nodes. T1 or T2. |
| Stage III-B | T3-4 N1 M0 | N1: Metastasis to 1 to 3 regional |
| | | lymph nodes. T3 or T4. |
| Stage III-C | any T, N2 M0 | N2: Metastasis to 4 or more regional |
| | | lymph nodes. Any T. |
| Stage IV | any T, any N, M1 | M1: Distant metastases present. |
| | | Any T, any N. |

In the context of the present invention, the patient is a subject suffering from colon cancer. In a particular embodiment, the colon cancer is any histology subtype which typically appears in colon cancer such as transitional cell carcinomas, squamous cell carcinoma and adenocarcinoma, any clinical subtype such as superficial, muscle-invasive or metastatic disease cancer and any TMN stage including T0-T4, N0-N2 and M0-M1 tumors. Furthermore, the present invention can also be applicable to a subject suffering from any stage of colon cancer (stages 0, IA, IB, IIA, IIB, IIIA, IIIB or IV). However, in a particular embodiment, the stage of colon cancer is II.

The term “subject”, as used herein, refers to all animals classified as mammals and includes, but is not restricted to, domestic and farm animals, primates and humans, e.g., human beings, non-human primates, cows, horses, pigs, sheep, goats, dogs, cats, or rodents. Preferably, the patient is a male or female human of any age or race.

In another particular embodiment, the patient has undergone surgical resection of the tumor. The term “surgical resection”, as used herein, includes either local excision whereby only the tumor and some tissue is removed or resection wherein part of all of the colon or the rectum is removed. Resection includes, without limitation, partial colectomy, right colectomy (Ileocolectomy), abdominoperineal resection, proctosigmoidectomy, total abdominal colectomy and total proctocolectomy.

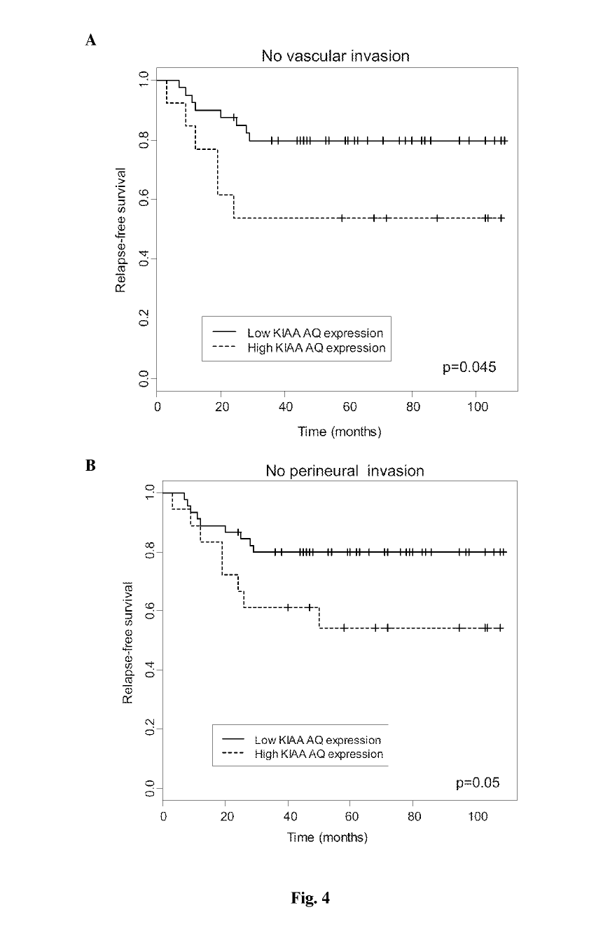

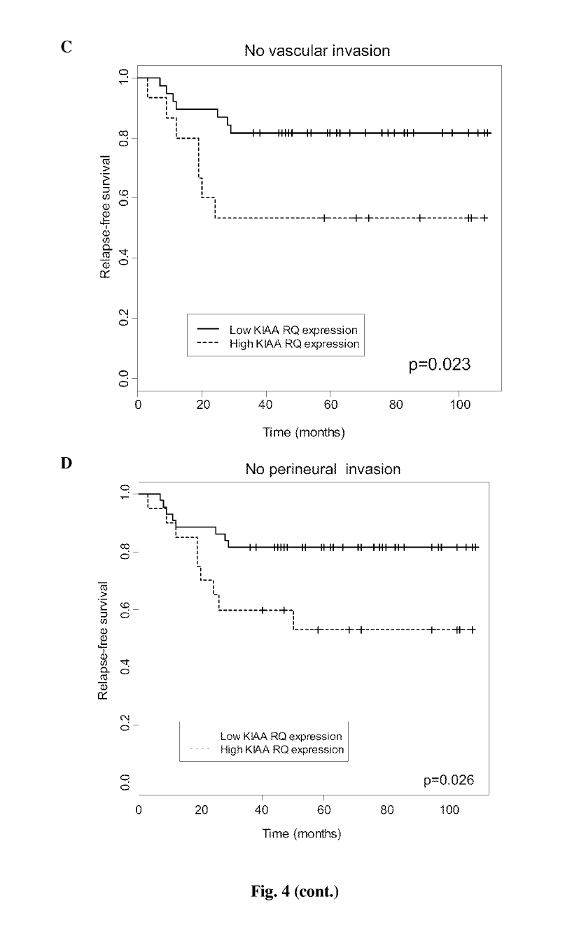

Several standard clinical risk factors have been described which are makers for prognosis of the disease such as T4 disease; tumor obstruction or perforation; poorly differentiated (grade 3) tumors; retrieval of <12 lymph nodes (ASCO recommendations); high preoperative CEA levels; vascular, lymphatic, and perineural invasion; and positive surgical margins in accordance with the College of American Pathologists consensus statement. Moreover, the inventors have found that patients with absence of vascular (FIGS. 4 A and C) or perineural invasion (FIGS. 4 B and D), i.e. cases with much better prognosis, showed a significant decrease in relapse-free survival if they over-expressed KIAA1456 (FIG. 4), suggesting that expression of KIAA1456 correlates with a worse clinical outcome even when standard risk factors indicate a good prognosis

Thus, in a preferred embodiment of the method of the invention, the patient does not show one or more of the above factors associated with clinical risk. In a still more preferred embodiment, the patient does not present intestinal neoplasic obstruction, vascular invasion and/or perineural invasion.

The expression “intestinal neoplasic obstruction” as used herein, is understood as a mechanical or functional obstruction of the intestines, preventing the normal transit of the products of digestion. It can occur at any level distal to the duodenum of the small intestine and is associated with a benign or malign neoplasm.

For neoplasm is understood as an abnormal mass of tissue as a result of neoplasia. Neoplasia is the abnormal proliferation of cells. The growth of this clone of cells exceeds, and is uncoordinated with, that of the normal tissues around it. It usually causes a lump or tumor. Neoplasms may be benign, pre-malignant or malignant.

Depending on the level of obstruction, bowel obstruction can present with abdominal pain, abdominal distension, vomiting, fecal vomiting, and constipation. Obstruction may be due to causes within the bowel lumen, within the wall of the bowel, or external to the bowel (such as compression, entrapment or volvulus). The main tools for measuring intestinal neoplasic obstruction are blood tests, X-rays of the abdomen, CT scanning and/or ultrasound. Radiological signs of bowel obstruction include bowel distension and the presence of multiple (more than six) gas-fluid levels on supine and erect abdominal radiographs. Contrast enema or small bowel series or CT scan can be used to define the level of obstruction, whether the obstruction is partial or complete, and to help define the cause of the obstruction. Colonoscopy, small bowel investigation with ingested camera or push endoscopy, and laparoscopy are other diagnostic options.

The expression “vascular invasion”, as used herein, is understood as the presence of malignant cells within endothelial cell—lined blood vessels beyond the muscularis propria. It is possible to detect extramural vascular invasion with MRI.

The expression “perineural invasion” as used herein, is understood as the growth of a tumor along the nerve (perineural). The infiltration of perineural spaces by a malign tumour (generally a carcinoma) indicates that the tumour is infiltrating and invades a low resistance space between peripheral nerve and connective tissue surrounding. Perineural invasion status can be determined at first glance by optic microscopy through examining haematoxylin and eosin-stained histological sections.

In a first step, the first method of the invention involves the determination of the expression level of the KIAA1456 gene in a sample from the patient.

The term “KIAA1456 gene” as used in the present invention, refers to the human KIAA1456 gene as well to the orthologues thereof from other species like chimpanzee (XM_{—}001138361.1 SEQ ID NO: 1, XM_{—}001138208.1 SEQ ID NO: 2, XM_{—}001138449.1 SEQ ID NO: 3) dog (XM_{—}540002.2 SEQ ID NO: 4), mouse (NM_{—}176952.4 SEQ ID NO: 5), rat (NM_{—}001107314 SEQ ID NO: 6) and the like. However, in a preferred embodiment, the KIAA1456 gene which is determined in the methods according to the present invention is the human KIAA1456 gene.

The human KIAA1456 gene (FLJ36980 or MGC43113) is located in chromosome 8 in the 8p22 region. It has two transcript variants or splice variants which results from alternative splicing of a single pre-mRNA, giving rise to two protein isoforms. The first transcript variant (transcript variant 1) (NM_{—}020844.2 SEQ ID NO:7) encode the isoform 1 (NP_{—}065895.2 SEQ ID NO: 8), chosen as the canonical sequence, has 454 amino acids, its function is still unknown although at structural level it has a homology domain with methyltransferase activity. The second transcript variant (transcript variant 2) (NM_{—}001099677.1, SEQ ID NO: 9) encode the isoform 2 (NP_{—}001093147.1, SEQ ID NO: 10) that has multiple differences with respect to isoform 1 due to the absence of several exons at its 5′ end. These differences produce a smaller 5′UTR region and cause a translation origin behind the start codon of variant 1, giving rise to a smaller protein of 328 amino acids which lacks the first 127 amino acids at the N-terminal end. The function of this isoform without having functional homology domains is also unknown. In a preferred embodiment, the invention involves the determining the expression level of KIAA1456 isoform 1.

The term “sample” as used herein, relates to any sample which can be obtained from the patient. The present method can be applied to any type of biological sample from a patient, such as a biopsy sample, FNAB (fine needle aspiration biopsy), a tissue, cell or fluid (serum, saliva, semen, sputum, cerebral spinal fluid (CSF), tears, mucus, sweat, milk, brain extracts, bronchial lavage, exfoliated epithelial cells obtained from feces as described by Nechvatal et al. (J. Microbiol. Methods., 2008, 72: 124-132) and the like. In a particular embodiment, said sample is a tumour tissue sample or portion thereof. In a more particular embodiment, said tumor tissue sample is a colorectal tumor tissue sample from a patient suffering from colorectal cancer. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology. In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze.

The method can be used on patients suffering from colorectal cancer in any of the stages of the tumor. In a preferred embodiment the patient is a patient suffering a type II colon cancer.

The term “expression level”, as used herein, refers to the value of a parameter which measures the extent of expression of a given gene. Said value can be determined by measuring the level of mRNA encoded by the gene of interest or by measuring the amount of protein encoded by said gene.

The expression level of the KIAA1456 gene can be determined by any method known to the skilled person including determining the level of the KIAA1456 mRNA or of the protein encoded by the KIAA1456 gene.

In a preferred embodiment, the determination of the expression level of the KIAA1456 gene can be carried out by measuring the expression level of the mRNA encoded by the KIAA1456 gene. For this purpose, the biological sample may be treated to physically, chemically or mechanically disrupt tissue or cell structure, to release intracellular components into an aqueous or organic solution to prepare nucleic acids for further analysis. The nucleic acids are extracted from the sample by procedures known to the skilled person and commercially available. RNA is then extracted from frozen or fresh samples by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

In a particular embodiment, the expression level is determined using mRNA obtained from a formalin-fixed, paraffin-embedded tissue sample. mRNA may be isolated from an archival pathological sample or biopsy sample which is first de-paraffinized. An exemplary de-paraffinization method involves washing the paraffinized sample with an organic solvent, such as xylene, for example. De-paraffinized samples can be rehydrated with an aqueous solution of a lower alcohol. Suitable lower alcohols, for example include, methanol, ethanol, propanols, and butanols. De-paraffinized samples may be rehydrated with successive washes with lower alcoholic solutions of decreasing concentration, for example. Alternatively, the sample is simultaneously de-paraffinized and rehydrated. The sample is then lysed and RNA is extracted from the sample.

Determination of the levels of the KIAA1456 mRNA can be carried out by any method known in the art such as RT-PCR followed with qPCR, northern blot, RNA dot blot, TaqMan, tag based methods such as serial analysis of gene expression (SAGE) including variants such as LongSAGE and SuperSAGE. Determination of the levels of the KIAA1456 mRNA can also be carried out by Fluorescence In Situ Hybridization, including variants such as Flow-FISH, qFiSH and double fusion fish (D-FISH) as described in WO2010030818, Femino et al. (Science, 1998, 280:585-590), Levsky et al. (Science, 2002, 297:836-840) or Raj et al. (PLoS Biology, 2006, 4:e309).

In a preferred embodiment, the gene mRNA expression level is often determined by reverse transcription polymerase chain reaction (RT-PCR). The detection can be carried out in individual samples or in tissue microarrays. In a still more preferred embodiment, the expression level of the KIAA1456 gene is determined by measuring the mRNA level of the transcript variant 1 of the human KIAA1456 gene.

In order to normalize the values of mRNA expression among the different samples, it is possible to compare the expression level of the mRNA of interest in the test samples with the expression of a control RNA. A “Control RNA” as used herein, relates to a RNA whose expression level does not change or changes only in limited amounts in tumor cells with respect to non-tumorigenic cells. Preferably, the control RNA are mRNA derived from housekeeping genes and which code for proteins which are constitutively expressed and carry out essential cellular functions. Preferred housekeeping genes for use in the present invention include β-2-microglobulin, GAPDH, PSMB4 (proteasome subunit, beta type, 4), ubiquitin, transferrin receptor, 18-S ribosomal RNA, cyclophilin, tubulin, β actin and tyrosine 3-monooxygenase/tryptophan 5-monooxygenase activation protein (YWHAZ). In a preferred embodiment, the control RNA is β-2-microglobulin mRNA. In one embodiment relative gene expression quantification is calculated according to the comparative Ct method using β-2-microglobulin, ribosomal 18S RNA, GAPDH and PSMB4 as endogenous gene controls and RNA controls (normal tissue from healthy donors and/or normal tissue adjacent to tumor from the same patient) as calibrators. Final results, are determined according to the formula 2-(ΔCt sample-ΔCt calibrator), where ΔCT values of the calibrator and sample are determined by subtracting the CT value of the target gene from the value of the geometric mean of the three housekeeping genes used.

In another embodiment, the expression level of the KIAA1456 gene is determined by measuring the expression of the KIAA1456 protein. The determination of the expression level of the KIAA1456 protein can be carried out by immunological techniques such as e.g. ELISA, Western blot or immunofluorescence. Western blot is based on the detection of proteins previously resolved by gel electrophoresis under denaturing conditions and immobilized on a membrane, generally nitrocellulose by the incubation with an antibody specific and a developing system (e.g. chemoluminiscent). The analysis by immunofluorescence requires the use of an antibody specific for the target protein for the analysis of the expression and subcellular localization by microscopy. Generally, the cells under study are previously fixed with paraformaldehyde and permeabilised with a non-ionic detergent. ELISA is based on the use of antigens or antibodies labelled with enzymes so that the conjugates formed between the target antigen and the labelled antibody results in the formation of enzymatically-active complexes. Since one of the components (the antigen or the labelled antibody) are immobilised on a support, the antibody-antigen complexes are immobilised on the support and thus, it can be detected by the addition of a substrate which is converted by the enzyme to a product which is detectable by, e.g. spectrophotometry or fluorometry. This technique does not allow the exact localisation of the target protein or the determination of its molecular weight but allows a very specific and highly sensitive detection of the target protein in a variety of biological samples (serum, plasma, tissue homogenates, postnuclear supernatants, ascites and the like). In a preferred embodiment, the KIAA1456 protein is detected by immunohistochemistry (IHC) analysis using thin sections of the biological sample immobilised on coated slides. The sections are then deparaffinised, if derived from a paraffinised tissue sample, and treated so as to retrieve the antigen. The detection can be carried out in individual samples or in tissue microarrays.

Any antibody or reagent known to bind with high affinity to the target protein can be used for detecting the amount of target protein. It is preferred nevertheless the use of antibody, for example polyclonal sera, hybridoma supernatants or monoclonal antibodies, antibody fragments, Fv, Fab, Fab′ y F(ab′)2, ScFv, diabodies, triabodies, tetrabodies and humanised antibodies.

In yet another embodiment, the determination of KIAA1456 protein expression level can be carried out by constructing a tissue microarray (TMA) containing the patient samples assembled, and determining the expression level of KIAA1456 protein by immunohistochemistry techniques Immunostaining intensity can be evaluated by two different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, and low expression (1+) versus moderate (2+) and high (3+) expression, taking into account the expression in tumoral cells and the specific cut-off for each marker. As a general criterion, the cut-offs were selected in order to facilitate reproducibility, and when possible, to translate biological events.

The determination of the level of expression of the KIAA1456 protein needs to be correlated with the reference values which may correspond to the median or average value of protein expression level of KIAA1456 measured in a collection of samples from normal individuals (i.e. people with no diagnosis of colon cancer) or from normal tissue from the same patient (i.e. tissue without tumoral cells). Once this median value is established, the level of this marker expressed in tumor tissues from patients can be compared with this median value, and thus be assigned a level of “low” or “decreased”, “normal” or “the same” or “high or “increased”. In a particular embodiment, an increase in protein expression above the reference value of at least 1.1-fold, 1.5-fold, 5-fold, 10-fold, 20-fold, 30-fold, 40-fold, 50-fold, 60-fold, 70-fold, 80-fold, 90-fold, 100-fold or even more compared with the reference value is considered as “increased” expression. In a particular embodiment, a decrease in protein expression below the reference value of at least 0.9-fold, 0.75-fold, 0.2-fold, 0.1-fold, 0.05-fold, 0.025-fold, 0.02-fold, 0.01-fold, 0.005-fold or even less compared with the reference value is considered as “decreased” expression. The term “same expression level”, as used herein, refers to expression levels which are substantially unaltered with respect to the reference value. For instance, the expression in the sample under study is considered to be the same as in the reference sample when the levels differ by no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5%, no more than 6%, no more than 7%, no more than 8%, no more than 9%, no more than 10% or no more than the percentage value that is the same as the error associated to the experimental method used in the determination.

Due to inter-subject variability (e.g. aspects relating to age, race, etc.) it is very difficult (if not practically impossible) to establish absolute reference values for KIAA1456. Thus, in a particular embodiment, the reference values for “increased”, “decreased” or “the same” KIAA1456 expression are determined by calculating percentiles by conventional means involving the testing of a group of samples isolated from normal subjects (i.e. people with no diagnosis of colon cancer) for the expression level of the KIAA1456 gene. The “increased” level can then be assigned, preferably, to samples wherein expression level for the KIAA1456 gene is equal to or in excess of percentile 50 in the normal population, including, for example, expression level equal to or in excess to percentile 60 in the normal population, equal to or in excess to percentile 70 in the normal population, equal to or in excess to percentile 80 in the normal population, equal to or in excess to percentile 90 in the normal population, and equal to or in excess to percentile 95 in the normal population.

In a preferred embodiment, the expression level of the KIAA1456 gene is determined by measuring the protein level of the KIAA1456 Isoform I or by measuring the mRNA level of the transcript variant 1 of the KIAA1456 gene using any of the methods mentioned above. In a more preferred embodiment, the expression level of the KIAA1456 gene is determined by measuring the protein level of the human KIAA1456 Isoform I or by measuring the mRNA level of the transcript variant 1 of the human KIAA1456 gene.

Once the expression level of the KIAA1456 gene is determined, the first method of the invention involves comparing said expression level with a reference sample.

The determination of the expression level of the mRNA encoded by the KIAA1456 gene needs to be correlated with the reference values which correspond to the median value of the expression level of the mRNA encoded by the KIAA1456 gene measured in a collection of samples from normal patients (i.e. people with no diagnosis of colon cancer) or from normal tissue from the same patient (i.e. tissue without tumoral cells). In any case it can contain a different number of samples.

Once the comparison has been carried out, the first method of the invention allows taking a decision on the convenience to administer a therapy to a patient based on whether the expression level is altered with respect to said reference level.

The term “altered expression level” as used herein refer to an increased level or a decreased level in the expression activity of a nucleic acid sequence and/or quantity of protein resulting from the expression of the nucleic acid sequence in or via a sample, as compared to a reference level. Depending on whether the altered expression level is increased, decreased or the same with respect to the reference level, the expression level of the KIAA1456 gene can be assigned as being “low” or “decreased”, “normal” or “high or “increased” with respect to a reference sample. In a particular embodiment, an increase in the expression level of the mRNA above the reference value of at least 1.1-fold, 1.5-fold, 5-fold, 10-fold, 20-fold, 30-fold, 40-fold, 50-fold, 60-fold, 70-fold, 80-fold, 90-fold, 100-fold or even more compared with the reference value is considered as “increased” expression. In a particular embodiment, a decrease in the expression level of the mRNA below the reference value of at least 0.9-fold, 0.75-fold, 0.2-fold, 0.1-fold, 0.05-fold, 0.025-fold, 0.02-fold, 0.01-fold, 0.005-fold or even less compared with the reference value is considered as “decreased” expression.

In a preferred embodiment, increased expression level is indicative that a therapy is adequate for said patient. In another preferred embodiment, decreased expression level is indicative that a therapy is not adequate for said patient.

### Method for Predicting the Clinical Outcome of a Patient

In a second aspect, the invention relates to a method for predicting the clinical outcome of a patient suffering from colorectal cancer (hereinafter second method of the invention) comprising the determination of the expression level of the KIAA1456 gene in a sample from said patient wherein increased expression level of the KIAA1456 gene in said sample when compared with reference level are indicative of a poor clinical outcome or wherein the same or a decreased expression level of the KIAA1456 gene in said sample when compared with reference level are indicative of a good clinical outcome.

The expression “predicting the clinical outcome”, as used herein, can be done by using any endpoint measurements used in oncology and known to the skilled practitioner. Useful endpoint parameters to describe the evolution of a disease include:
disease-free survival which, as used herein, refers to the time interval from the date of the last course of chemotherapy to the date of recurrence, death or last follow-upoverall survival which, as used herein,
overall survival which, as used herein, refers to the time from entry in the study until death or censoring,
disease-free progression which, as used herein, describes the proportion of patients in complete remission who have had no recurrence of disease during the time period under study.
objective response, which, as used in the present invention, describes the proportion of treated people in whom a complete or partial response is observed.
tumor control, which, as used in the present invention, relates to the proportion of treated people in whom complete response, partial response, minor response or stable disease≧6 months is observed.
progression free survival which, as used herein, is defined as the time from start of treatment to the first measurement of cancer growth.
six-month progression free survival or PFS6″ rate which, as used herein, relates to the percentage of people wherein free of progression in the first six months after the initiation of the therapy and
median survival which, as used herein, relates to the time at which half of the patients enrolled in the study are still alive.

In an embodiment, the prediction of the clinical outcome is measured as disease-free survival and/or overall survival.

As will be understood by those skilled in the art, the prediction of the clinical outcome may usually not be correct for 100% of the subjects to be diagnosed. The term, however, requires that a statistically significant portion of subjects can be adequately prognosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% at least 95%. The p-values are, preferably, 0.2, 0.1, and 0.05.

In a first step, the first method of the invention comprises determining the expression level of the KIAA1456 gene in a sample from said patient.

The terms “expression level”, “patient”, “sample” “KIAA1456 gene”, “colorectal cancer”, have been described in detail above and are used in the same meaning in the context of the second method of the invention.

In a preferred embodiment, the expression level of the KIAA1456 gene is determined by measuring the level of mRNA encoded by the KIAA1456 gene or the level of KIAA1456 protein. In yet another embodiment, the expression level of KIAA1456 is determined by measuring the mRNA level of the transcript variant 1 of the human KIAA1456 gene. The determination can be carried out using any of the methods mentioned above.

In another embodiment, the sample wherein the determination of the expression level of the KIAA1456 gene is a tumor biopsy.

In another embodiment, the patient is a human patient. In another embodiment, the patient has undergone surgical resection of the tumor. In yet another embodiment, the patient suffers from stage II colorectal cancer. The terms “surgical resection” and “stage II” have been defined above.

In yet another embodiment of the method according to the invention is carried out in patients who do not show additional clinical risk factors. In a preferred embodiment, the patient does not show intestinal neoplasic obstruction, vascular invasion and/or perineural invasion.

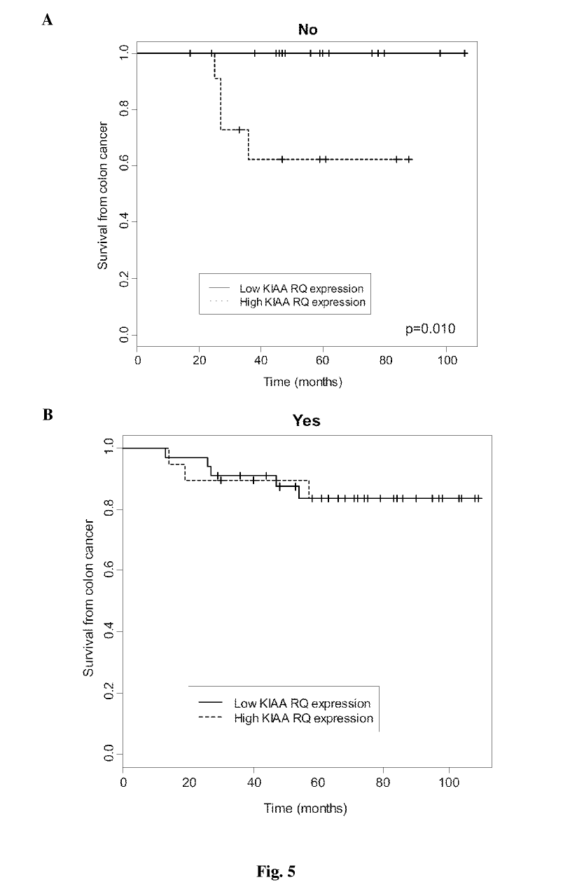

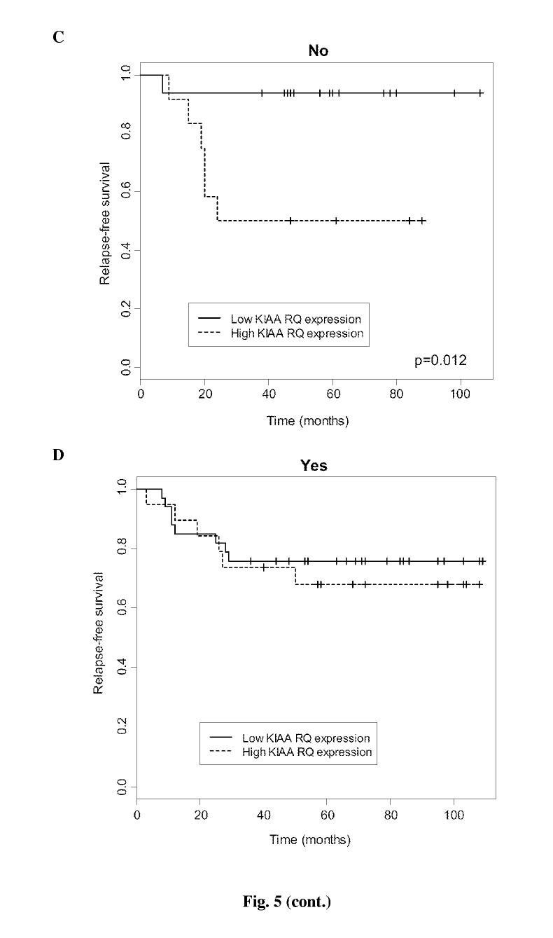

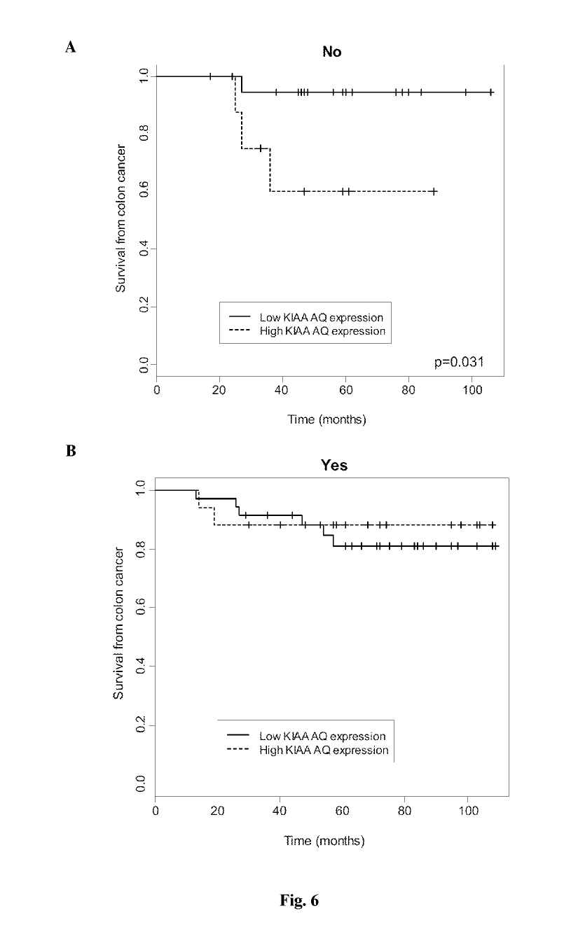

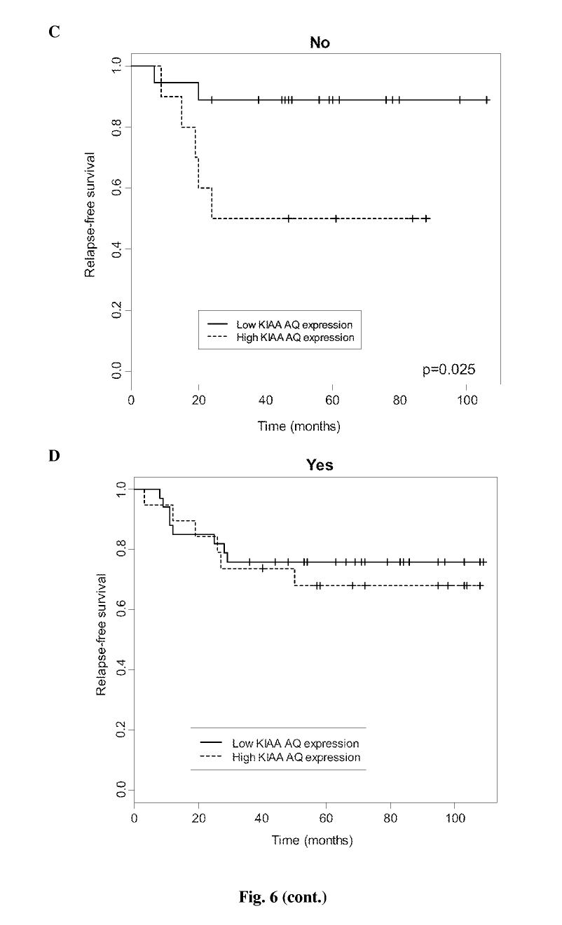

The authors of the present invention have observed that the value of the KIAA1456 gene expression level as predictive marker for the clinical outcome of patients suffering from colorectal cancer increases in those patients which have not been administered adjuvant chemotherapy or radiotherapy after surgery. As shown in example 4 of the present invention, the prognosis of the patients who are not treated with adjuvant chemotherapy is radically worse in the patients with overexpression of the gene (p=0.010 for overall survival and p=0.012 for relapse-free survival) (FIGS. 5A and 5C and 6A and 6C). These differences are not observed in the patients treated with chemotherapy in which the prognosis is much better for both cases (FIGS. 5B and 5D and 6B and 6D). Thus, in yet another embodiment, the patient has not been treated with neoadjuvant or adjuvant therapy.

The term “adjuvant therapy”, as used herein, relates to additional treatment, usually given after surgery where all detectable disease has been removed, but where there remains a statistical risk of relapse due to occult disease. The term “neoadjuvant therapy”, as used herein, refers to systemic drug treatment or radiation therapy given to people with cancer prior to surgery which aim is to reduce the size or extent of the cancer before receiving surgery, thus making procedures easier and more likely to be successful, and reducing the consequences of a more extensive surgery that would have to be done if the tumor wasn't reduced in size or extent.

In a second step, the second method according to the invention involves comparing the expression level of the KIAA1456 gene in the sample with reference level. The terms “expression level” and “reference level” have been described in detail above and are used in the context of the second method of the invention in the same context.

Lastly, the second method of the invention further comprises deciding whether the patient will have a poor clinical outcome or good clinical outcome based on the relative expression level of the KIAA1456 gene wherein an increased expression level of the KIAA1456 gene in said sample when compared with reference level are indicative of a poor clinical outcome or wherein the same or decreased expression level of the KIAA1456 gene in said sample when compared with reference level are indicative of a good clinical outcome.

The terms “decreased expression level”, “increased expression level”, “same expression level” and “reference level” have been described in detail above and are used in the context of the second method of the invention in the same context.

The term “poor clinical outcome” refers to any worsening, or increase in frequency of, clinical symptoms associated with the disorder, as determined using known diagnostic methods or using any endpoint measurements used in oncology and known to the skilled practitioner as defined above.

The term “positive clinical outcome” refers to any improvement, or decrease in frequency of, clinical symptoms associated with the disorder, as determined using known diagnostic methods or using any endpoint measurements used in oncology and known to the skilled practitioner as defined above.

### Kits and Uses Thereof

In another aspect, the invention relates to a kit useful in the implementation of the methodology described herein.

In the context of the present invention, “kit” is understood as a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components which are in the kit. Said instructions can be in the form of printed material or in the form of an electronic support capable of storing instructions such that they can be read by a subject, such as electronic storage media (magnetic disks, tapes and the like), optical media (CD-ROM, DVD) and the like. Additionally or alternatively, the media can contain Internet addresses that provide said instructions.

Thus, the kit of the invention comprises a set of agents capable of specifically detecting the expression level of KIAA1456 and, optionally, a reagent for detecting a housekeeping gene or the protein encoded by said housekeeping gene. In a particular embodiment, the isoform from KIAA1456 detected is the isoform I.

“Reagent which allows determining the expression level of a gene” means a compound or set of compounds that allows determining the expression level of a gene both by means of the determination of the level of mRNA or by means of the determination of the level of protein. Thus, reagents of the first type include probes capable of specifically hybridizing with the mRNAs encoded by said genes. Reagents of the second type include compounds that bind specifically with the proteins encoded by the marker genes and preferably include antibodies, although they can be specific aptamers. In a preferred embodiment, the reagent which allows determining the expression level of the KIAA1456 gene form at least comprise at least 50%, at least 55%, at least 60%, at least 65%, at least 70%, at least 75%, at least 80%, at least 90%, at least 95%, at least 96%, at least 97%, at least 98% or at least 99% of the total amount of reagents forming the kit.

In a particular embodiment of the kit of the invention, the reagents of the kit is a nucleic acid which is capable of specifically detecting the mRNA level of KIAA1456 and/or or the level of KIAA1456 protein. Nucleic acids capable of specifically hybridizing with the KIAA1456 gene can be one or more pairs of primer oligonucleotides for the specific amplification of fragments of the mRNAs (or of their corresponding cDNAs) of said gene.

In a preferred embodiment, the first component of the kit of the invention comprises a probe which can specifically hybridize to the KIAA1456 gene.

The probes included in the kit that are capable of hybridizing with the nucleic acids can be nucleic acids or analogs thereof which maintain the hybridization capacity such as for example, nucleic acids in which the phosphodiester bond has been substituted with a phosphorothioate, methylimine, methylphosphonate, phosphoramidate, guanidine bond and the like, nucleic acids in which the ribose of the nucleotides is substituted with another hexose, peptide nucleic acids (PNA). The length of the probes can be of 7, 10, 15, 20, 25, 30, 35, 40, 45, 50, 55, 60, 65, 70, 75, 100 nucleotides and vary in the range of 10 to 1000 nucleotides, preferably in the range of 15 to 150 nucleotides, more preferably in the range of 15 to 100 nucleotides and can be single-strand or double-strand nucleic acids.

The selection of the specific probes for the different target genes is carried out such that they bind specifically to the target nucleic acid with a minimum hybridization to non-related genes. However, there are probes of 20 nucleotides which are not unique for a certain mRNA. Therefore, probes directed to said sequences will show a cross-hybridization with identical sequences that appear in mRNA of non-related genes. In addition, there are probes that do not specifically hybridize with the target genes in the conditions used (because of secondary structures or of interactions with the substrate of the array). This type of probe must not be included in the array. Therefore, the person skilled in the art will observe that the probes that are going to be incorporated in a certain array must be optimized before their incorporation to the array. The optimization of the probes is generally carried out by generating an array containing a plurality of probes directed to the different regions of a certain target polynucleotide. This array is put into contact firstly with a sample containing the target nucleic acid in an isolated form and, secondly, with a complex mixture of nucleic acids. Probes which show a highly specific hybridization with the target nucleic acid but low or no hybridization with the complex sample are thus selected for their incorporation to the arrays of the invention. Additionally, it is possible to include in the array hybridization controls for each of the probes that is going to be studied. In a preferred embodiment, the hybridization controls contain an altered position in the central region of the probe. In the event that high level of hybridization is observed between the studied probe and its hybridization control, the probe is not included in the array.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50 .degree. C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 .degree. C.; or (3) employ 50% formamide, 5 .times.SSC (0.75 M NaCl, 0.075 M sodium citrate), 50 mM sodium phosphate (pH 6.8), 0.1% sodium pyrophosphate, 5 .times. Denhardt's solution, sonicated salmon sperm DNA (50 .mu.g/ml), 0.1% SDS, and 10% dextran sulfate at 42 .degree. C., with washes at 42 .degree. C. in 0.2 .times.SSC (sodium chloride/sodium citrate) and 50% formamide, followed by a high-stringency wash consisting of 0.1 .times.SSC containing EDTA at 55 .degree. C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37 .degree. C. in a solution comprising: 20% formamide, 5 .times.SSC (150 mM NaCl, 15 mM trisodium citrate), 50 mM sodium phosphate (pH 7.6), 5 .times. Denhardt's solution, 10% dextran sulfate, and 20 mg/ml denatured sheared salmon sperm DNA, followed by washing the filters in 1 .times.SSC at about 37-50 .degree. C. The skilled artisan will recognize how to adjust the temperature, ionic strength, etc. as necessary to accommodate factors such as probe length and the like.

In another preferred embodiment, the probes or antibodies forming the kit of the invention are coupled to an array.

The microarrays comprise a plurality of nucleic acids that are spatially distributed and stably associated to a support (for example, a biochip). The nucleic acids have a sequence complementary to particular subsequences of genes the expression of which is to be detected, therefore are capable of hybridizing with said nucleic acids. In the methods of the invention, a microarray comprising an array of nucleic acids is put into contact with a preparation of nucleic acids isolated from the patient object of the study. The incubation of the microarray with the preparation of nucleic acids is carried out in conditions suitable for the hybridization. Subsequently, after the elimination of the nucleic acids which have not been retained in the support, the hybridization pattern is detected, which provides information on the genetic profile of the sample analyzed. Although the microarrays are capable of providing both qualitative and quantitative information of the nucleic acids present in a sample, the invention requires the use of arrays and methodologies capable of providing quantitative information.

The invention contemplates a variety of arrays with regard to the type of probes and with regard to the type of support used

In a preferred embodiment, the array contains a plurality of probes complementary to subsequences of the target nucleic acid of a constant length or of variable length in a range of 5 to 50 nucleotides. The array can contain all the specific probes of a certain mRNA of a certain length or can contain probes selected from different regions of an mRNA. Each probe is assayed in parallel with a probe with a changed base, preferably in a central position of the probe. The array is put into contact with a sample containing nucleic acids with sequences complementary to the probes of the array and the signal of hybridization with each of the probes and with the corresponding hybridization controls is determined Those probes in which a higher difference is observed between the signal of hybridization with the probe and its hybridization control are selected. The optimization process can include a second round of optimization in which the hybridization array is hybridized with a sample that does not contain sequences complementary to the probes of the array. After the second round of selection, those probes having signals of hybridization lower than a threshold level will be selected. Thus, probes which pass both controls, i.e., which show a minimum level of unspecific hybridization and a maximum level of specific hybridization with the target nucleic acid are selected.

The microarrays of the invention contain not only specific probes for the polynucleotides indicating a determined pathophysiological situation, but also containing a series of control probes, which can be of three types: normalization controls, expression level controls and hybridization controls

Normalization controls are oligonucleotides that are perfectly complementary to labeled reference sequences which are added to the preparation of nucleic acids to be analyzed. The signals derived from the normalization controls after the hybridization provide an indication of the variations in the hybridization conditions, intensity of the marker, efficiency of the detection and another series of factors that can result in a variation of the signal of hybridization between different microarrays. The signals detected from the rest of probes of the array are preferably divided by the signal emitted by the control probes, thus normalizing the measurements. Virtually any probe can be used as normalization control. However, it is known that the efficiency of the hybridization varies according to the composition of nucleotides and the length of the probe. Therefore, preferred normalization probes are those which represent the mean length of the probes present in the array, although they can be selected such that they include a range of lengths that reflect the rest of probes present in the array. The normalization probes can be designed such that they reflect the mean composition of nucleotides of the rest of probes present in the array. A limited number of normalization probes are preferably selected such that they hybridize suitably, i.e., they do not have a secondary structure and do not show sequence similarity with any of the probes of the array is used. The normalization probes can be located in any position in the array or in multiple positions in the array to efficiently control variations in hybridization efficiency related to the structure of the array. The normalization controls are preferably located in the corners of the array and/or in the center thereof.

The expression controls are probes which hybridize specifically with genes which are expressed constitutively in the sample which is analyzed. The expression level controls are designed to control the physiological state and the metabolic activity of the cell. The examination of the covariance of the expression level control with the expression level of the target nucleic acid indicates if the variations in the expression level are due to changes in the expression level or are due to changes in the overall transcriptional rate in the cell or in its general metabolic activity. Thus, in the case of cells which have deficiencies in a certain metabolite essential for cell viability, the observation of a decrease both in the expression level of the target gene as in the expression level of the control is expected. On the other hand, if an increase in the expression of the expression of the target gene and of the control gene is observed, it probably due to an increase of the metabolic activity of the cell and not to a differential increase in the expression of the target gene. Any probe corresponding to a gene expressed constitutively, such as genes encoding proteins which exert essential cell functions such as β-2-microglobulin, ubiquitin, ribosomal protein 18S, cyclophilin A, tyrosine 3-monooxygenase/tryptophan 5-monooxygenase activation protein (YWHAZ), PSMB4 (proteasome subunit, beta type, 4), transferrin receptor, tubulin, beta-actin, GAPDH and the like, can be used. In a preferred embodiment, the expression level controls are GAPDH, tyrosine 3-monooxygenase/tryptophan 5-monooxygenase activation protein (YWHAZ), ribosomal protein 18S, ubiquitin, beta-actin and β-2-microglobulin.

Hybridization controls can be included both for the probes directed to target genes and for the probes directed to the expression level or to the normalization controls. Error controls are probes of oligonucleotides identical to the probes directed to target genes but which contain mutations in one or several nucleotides, i.e., which contain nucleotides in certain positions which do not hybridize with the corresponding nucleotide in the target gene. The hybridization controls are selected such that, applying the suitable hybridization conditions, the target gene should hybridize with the specific probe but not with the hybridization control or with a reduced efficiency. The hybridization controls preferably contain one or several modified positions in the center of the probe. The hybridization controls therefore provide an indication of the degree of unspecific hybridization or of cross-hybridization to a nucleic acid in the sample to a probe different from that containing the exactly complementary sequence.

The arrays of the invention can also contain amplification and sample preparation controls which are probes complementary to subsequences of selected control genes because they normally do not appear in the biological sample object of the study, such as probes for bacterial genes. The RNA sample is supplemented with a known amount of a nucleic acid which hybridizes with the selected control probe. The determination of the hybridization to said probe indicates the degree of recovery of the nucleic acids during their preparation as well as an estimation of the alteration caused in the nucleic acids during the processing of the sample.

Once a set of probes showing the suitable specificity and a set of control probes are provided, the latter are arranged in the array in a known position such that, after the steps of hybridization and of detection, it is possible to establish a correlation between a positive signal of hybridization and the particular gene from the coordinates of the array in which the positive signal of hybridization is detected.

The microarrays can be high density arrays with thousands of oligonucleotides by means of photolithographic in situ synthesis methods (Fodor et al., 1991, Science, 767-773). This type of probe is usually redundant, i.e., they include several probes for each mRNA which is to be detected. In a preferred embodiment, the arrays are low density arrays or LDA containing less than 10000 probes per square centimeter. In said low density arrays, the different probes are manually applied with the aid of a pipette in different locations of a solid support (for example, a crystal surface, a membrane). The supports used to fix the probes can be obtained from a large variety of materials, including plastic, ceramics, metals, gels, membranes, crystals and the like. The microarrays can be obtained using any methodology known for the person skilled in the art.

After the hybridization, in the cases in which the non-hybridized nucleic acid is capable of emitting a signal in step of detection, a step of washing is necessary to eliminate said non-hybridized nucleic acid. The step of washing is carried out using methods and solutions known by the person skilled in the art.

In the event that the labeling in the nucleic acid is not directly detectable, it is possible to connect the microarray comprising the target nucleic acids bound to the array with the other components of the system necessary to cause the reaction giving rise to a detectable signal. For example, if the target nucleic acids are labeled with biotin, the array is put into contact with conjugated streptavidin with a fluorescent reagent in suitable conditions so that the binding between biotin and streptavidin occurs. After the incubation of the microarray with the system generating the detectable signal, it is necessary to carry out a step of washing to eliminate all the molecules which have bound non-specifically to the array. The washing conditions will be determined by the person skilled in the art using suitable conditions according to the system generating the detectable signal and which are well known for the person skilled in the art.

The resulting hybridization pattern can be viewed or detected in several different ways, said detection being determined by the type of system used in the microarray. Thus, the detection of the hybridization pattern can be carried out by means of scintillation counting, autoradiography, determination of a fluorescent signal, calorimetric determinations, detection of a light signal and the like.

Prior to the step of detection, it is possible to treat the microarrays with a specific endonuclease for single-strand DNA, such that the DNA that has bound non-specifically to the array is eliminated whereas the double-strand DNA resulting from the hybridization of the probes of the array with the nucleic acids of the sample object of study remains unchanged. Endonucleases suitable for this treatment include the Si nuclease, mung bean nuclease and the like. In the event that the treatment with endonuclease is carried out in an assay in which the target nucleic acid is not labeled with a directly detectable molecule (for example, in an assay in which the target nucleic acid is biotinylated), the treatment with endonuclease will be carried out before putting the microarray into contact with the other members of the system producing the detectable signal.

After the hybridization and the possible subsequent washing and treatment processes, the hybridization pattern is detected and quantified, for which the signal corresponding to each point of hybridization in the array is compared to a reference value corresponding to the signal emitted by a known number of terminally labeled nucleic acids in order to thus obtain an absolute value of the number of copies of each nucleic acid which is hybridized in a certain point of the microarray.

In the event that the expression level of the KIA1456 protein is to be determined, the kit of the invention comprises at least one specific antibody for said protein.

For this purpose, the arrays of antibodies such as those described by De Wildt et al. (2000) Nat. Biotechnol. 18:989-994; Lueking et al. (1999) Anal. Biochem. 270:103-111; Ge et al. (2000) Nucleic Acids Res. 28, e3, I-VII; MacBeath and Schreiber (2000) Science 289:1760-1763; WO 01/40803 and WO 99/51773A1 are useful. The antibodies of the array include any immunological agent capable of binding to a ligand with high affinity, including IgG, IgM, IgA, IgD and IgE, as well as molecules similar to antibodies which have an antigen binding site, such as Fab′, Fab, F(ab′)2, single domain antibodies or DABS, Fv, scFv and the like. The techniques for preparing said antibodies are very well known for the person skilled in the art and include the methods described by Ausubel et al. (Current Protocols in Molecular Biology, eds. Ausubel et al, John Wiley & Sons (1992)).

The antibodies of the array can be applied at high speed, for example, using commercially available robotic systems (for example, those produced by Genetic Microsystems or Biorobotics). The substrate of the array can be nitrocellulose, plastic, crystal or can be of a porous material as for example, acrylamide, agarose or another polymer. In another embodiment, it is possible to use cells producing the specific antibodies for detecting the proteins of the invention by means of their culture in array filters. After the induction of the expression of the antibodies, the latter are immobilized in the filter in the position of the array where the producing cell was located.

An array of antibodies can be put into contact with a labeled target and the binding level of the target to the immobilized antibodies can be determined. If the target is not labeled, a sandwich type assay can be used in which a second labeled antibody specific for the polypeptide which binds to the polypeptide which is immobilized in the support is used. The quantification of the amount of polypeptide present in the sample in each point of the array can be stored in a database as an expression profile. The array of antibodies can be produced in duplicate and can be used to compare the binding profiles of two different samples.

Antibodies, or a fragment thereof, capable of detecting an antigen, capable of specifically binding to KIAA1456 protein or to variants thereof are, for example, monoclonal and polyclonal antibodies, antibody fragments, Fv, Fab, Fab′ y F(ab′)2, ScFv, diabodies, triabodies, tetrabodies and humanised antibodies.

In a preferred embodiment, the reagents of the kit are DNA or RNA probes or antibodies.

Said reagents, specifically, the probes and the antibodies, may be fixed onto a solid support, such as a membrane, a plastic or a glass, optionally treated in order to facilitate fixation of said probes or antibodies onto the support. Said solid support, which comprises, at least, a set of antibodies capable of specifically binding to KIAA1456 protein or to variants thereof, and/or probes specifically hybridized with the KIAA1456 gene, may be used for the detection of the expression level by means of array technology.

The kits of the invention optionally comprise additional reagents for detecting the polypeptide encoded a housekeeping gene or the mRNA encoded by said housekeeping genes. The availability of said additional reagent allows the normalization of measurements taken in different samples (e.g. the test sample and the control sample) to exclude that the differences in expression of the different biomarkers are due to a different amount of total protein in the sample rather than to real differences in relative expression level. More than one housekeeping gene can be used. Housekeeping genes, as used herein, relate to genes which code for proteins which are constitutively expressed and carry out essential cellular functions. Preferred housekeeping genes for use in the present invention include β-2-microglobulin, GAPDH, PSMB4 (proteasome subunit, beta type, 4), ubiquitin, transferrin receptor, 18-S ribosomal RNA, cyclophilin, tubulin, β actin and tyrosine 3-monooxygenase/tryptophan 5-monooxygenase activation protein (YWHAZ).

In another embodiment, the invention relates to the use of a for predicting the clinical outcome of a subject suffering from colorectal cancer, wherein if said agents detect a high expression level of KIAA1456 gene, with respect to reference values, then the clinical outcome of the subject is poor.

Methods for detecting the expression level of KIAA1456 and the methods for determining as well as the standard reference values has been described previously.

In another aspect, the invention relates to the use of a kit of the invention for the diagnosis of colorectal cancer or for the determination of the stage of a colorectal tumor.

The following examples are provided as merely illustrative and are not to be construed as limiting the scope of the invention.

### EXAMPLES

### Methodology

### Samples of Patients

The analysis of the mRNA expression level of KIAA1456 gene isoform 1 was conducted in a total of 98 stage II colorectal cancer tumor tissue samples from patients who underwent surgery in the Hospital Universitario de La Paz of Madrid, between the years 2000 and 2005. The ethics committee of the Hospital La Paz approved this study. The Pathology Department of the Hospital Universitario de La Paz examined and determined the stage at local level of the samples according to the criteria established by the AJCC and the UICC. A total of 80 samples were selected for the subsequent statistical analysis thereof due to having good-quality RNA. A pool of 10 samples of normal tissue adjacent to some of the tumors included in the study and a pool of 4 samples of normal tissue were used as normal tissue references.

The clinicopathological variables of these 80 cases were determined following well-established criteria and are summarized in Table 2.

TABLE 2
Total n = 80

| Parameter | mean ± SD |
|---|---|
| Age (years) | 67.80 ± 10.63 |
| Sex | n (%) |
| Men | 45 (56.3) |
| Women | 35 (43.8) |
| Primary tumor (T), | n (%) |
| T3 | 57 (71.2) |
| T4 | 22 (27.5) |
| No data available | 1 (1.3) |
| Perineural invasion | n (%) |
| NO | 63 (78.8) |
| YES | 14 (17.5) |
| No data available | 3 (3.7) |
| Intestinal obstruction | n (%) |
| NO | 69 (86.2) |
| YES | 11 (13.8) |
| Desmoplasia | n (%) |
| NO | 37 (46.2) |
| YES | 25 (31.3) |
| No data available | 18 (22.5) |
| Histopathological grade | n (%) |
| G1 | 5 (6.3) |
| G2 | 69 (86.2) |
| G3 | 5 (6.3) |
| No data available | 1 (1.2) |
| Type of colorectal cancer, n (%) | n (%) |
| Colon | 41 (51) |
| Rectal | 25 (31) |
| Sigma | 14 (17) |
| Vascular invasion | n (%) |
| NO | 53 (66.2) |
| YES | 24 (30) |
| No data available | 3 (3.8) |
| Chemotherapy¹ | n (%) |
| NO | 28 (35) |
| YES | 52 (65) |
| Recurrence | n (%) |
| NO | 59 (73.8) |
| YES | 21 (26.2) |

¹46 patients received 6 cycles of UFT-LV and 6 patients received xeloda

The periods of observations of the patients ranged from 3 months to 109 months with a median follow-up of 59 months. At the time of the analysis, 21 of the 80 patients had presented relapse (26.2%) and 12 of them (15%) had died.

A total of 28 patients (35%) did not receive adjuvant chemotherapy, the rest of them (52, 65%) received monotherapy with a fluoropyrimidine (UFT-LV or xeloda).

Isolation of Total RNA from Paraffined Human Tissue Samples

For the extraction of the total RNA from the paraffined clinical samples, the starting material was 10 sections of 7 microns per piece. As a step prior to isolating the RNA, the tissue sections were deparaffinized and rehydrated by means of passes through xylol and decreasing alcohols (100%, 90% and 70%). Then, to isolate the RNA, the MasterPure RNA purification kit (Epicentro) was used according to the manufacturer's indications which, in addition to the purification of the RNA, include a treatment with DNAse to eliminate any trace of contaminating genomic DNA.

Reverse Transcription and Real-Time PCR (qPCR)

To obtain cDNA, the starting material was 1 μg of the total RNA from tissue samples of patients. Reverse transcription was carried out at 37° C. for 2 hours using the High-Capacity cDNA Archive kit (Applied Biosystems). Each cDNA was then analyzed in triplicate using the ABI PRISM 7700 Sequence Detector (Applied Biosystems). PCR was carried out using the Taqman Universal PCR reaction mixture (Applied Biosystems), which contained the reagent ROX to standardize the emissions. The probes used for the specific amplification of KIAA1456 gene isoform 1 were purchased from Applied Biosystems as Taqman Gene Expression Assay (assay ID: Hs00332747_m1). By means of the information available from Applied Biosystems it was verified that said probe only recognized isoform 1 and not isoform 2.

The β-2 microglobulin (ID Applied Biosystems: Hs99999907_m1), PSMB4 (ID Applied Biosystems: Hs00160598_ml) or GAPDH (ID Applied Biosystems: Hs99999905_m1) genes were amplified as internal controls. As the stability of the three genes is very high, the geometric mean of the three genes was selected as normalization factor. The PCT temperature cycles were: 10 minutes at 95° C., 40 cycles of 15 seconds at 95° C. and 1 minute at 60° C.

The relative quantification of KIAA1456 expression was calculated by both the 2^{-ΔΔCt} method and by the 2^{-ΔCt} method [Livak K J, Schmittgen T D: Analysis of relative gene expression data using real-time quantitative PCR and the 2(-delta delta c(t)) method. Methods (San Diego, Calif. 2001; 25:402-408]. The 2^{-ΔΔCt} method (User Bulletin No. 2 of Applied Biosystems (P/N 4303859)), which will hereinafter be referred to as “RQ”, consists of calculating the standardized KIAA1456 gene expression in the tumor tissue sample with respect to the standardized expression of the same gene in the reference sample which in this case is the adjacent normal tissue samples. The data are presented as the “change in times of expression” (RQ) of KIAA1456 standardized with the geometric mean of reference genes and relative to a control normal tissue sample.

The 2^{-ΔCt} method, which will hereinafter be referred to as “AQ”, consists of using the standardized levels of the tumor samples without relating them to the control normal tissue sample. The data are shown as the relative levels of the messenger RNA of the study gene standardized with the geometric mean of reference genes and multiplied by 10².

### Statistical Analysis

Data management was conducted by means of the computer software SPSS, version 15.0 (Inc, Chicago, Ill.) and all analyses were carried out with the R freeware statistical package (version 2.9.1) (R Development Core Team, 2009).

Whereas survival was defined as the time passed from the diagnosis up to the death, relapse-free survival was understood as the time passed from the diagnosis up to the first relapse (in all the cases exists right censoring, since not all the patients die they all do not relapse either).

For each gene and both, survival and relapse-free survival, a cut-off in the gene expression was determined as that point of the (normalized) expression of the gene in which the predictive value was maximized. There was understood that this was achieved when the area under the ROC curve (AUC) was the maximum, which would provide the ideal combination of sensibility and of specificity.

ROC curves were constructed using, in each case, the indicator of death (or relapse) and the expected value obtained from an Andersen-Gill model (AG model) [Andersen P K, Gill R D. Annals of Statistics 1982; 10:1100-1120; Andersen P K. et al; Statistical Models based on Counting Processes. New York: Springer-Verlag, 1993]. In previous stages of the analysis it was observed that, for any of the categorizations of the values of the gene expression (especially, in ‘under’ and ‘over’ expression) as well as for the categories of determined covariates (especially age and pathological grade), the risks might not be proportional. For this motive, and in order to obtain consistent estimates, we used AG models. This model allows the evaluation of time-dependent variables that could be explanatory of the risk. In this case, the estimation of the curves of survival is done by means of the Aalen-Johansen's estimate, constructed in turn from the Nelson-Aalen's estimate of the accumulated intensities of transition [Andersen P K. et al; Statistical Models based on Counting Processes. New York: Springer-Verlag, 1993]. Borgan [Borgan O. Encyclopaedia of Biostatistics. John Wiley & Sons, 1998] shows that this estimate is not any more than a matrix version of the Kaplan-Meier's estimate.

Then, we represented, for all the samples with (normalized) gene expression values above and below the cut-off, the survival curves (both, overall and relapse-free survival), stratifying by the covariates of interest. The statistical difference between these curves was tested by means of the Harrington and Fleming's G-rho family [Harrington D P, Fleming T R. Biometrika 1982; 69:553-566].

To assess the effect of the gene expression on the survival controlling for possible confounders, several AG models [Andersen P K, Gill R D. Annals of Statistics 1982; 10:1100-1120; Andersen P K. et al; Statistical Models based on Counting Processes. New York: Springer-Verlag, 1993] were fitted. In all cases, we controlled for the presence of false positives by means of non-parametric bootstrapping [Efron B, Tibshirani R. An introduction to the Bootstrap. Nueva York, London: Chapman and Hall, 1993].

### Example 1

### Transcriptional Expression Levels of KIAA1456 Gene Isoform 1 in Patients with Stage II Colorectal Cancer

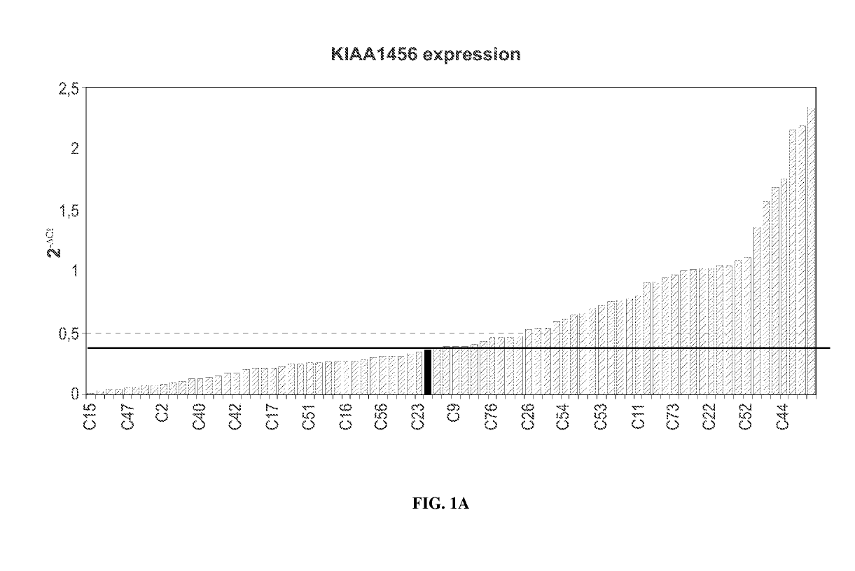

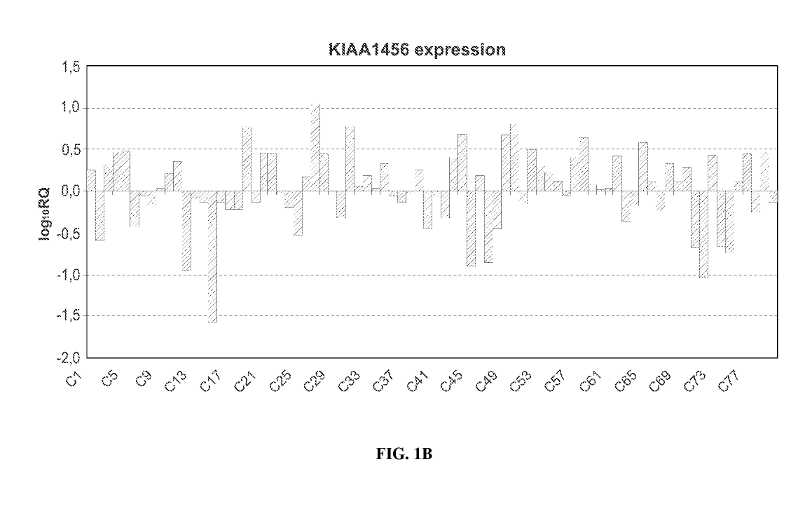

After carrying out the quantitative PCR, samples having a Ct of the β2-microglobulin control gene of less than 27.3 were selected, since greater values indicate a poor RNA quality. Finally, 80 of the 98 available samples presented a good-quality endogenous gene amplification and were the ones analyzed to study the transcriptional levels of KIAA1456 (FIG. 1A). By comparing the tumor levels with respect to the pool of normal tissues used as reference, a significant silencing was found in 17 cases (21%, RQ<0.5), it was also detected that in 30 samples there was a significant overexpression (38%, RQ>1,5) with respect to the control normal tissue, whereas in the rest of the cases (33, i.e., 41%) there were no changes with respect to the normal tissue (FIG. 1B).

### Example 2

### Correlation Between the Clinicopathological Parameters and the Expression Levels of KIAA1456 in Tumor Tissues

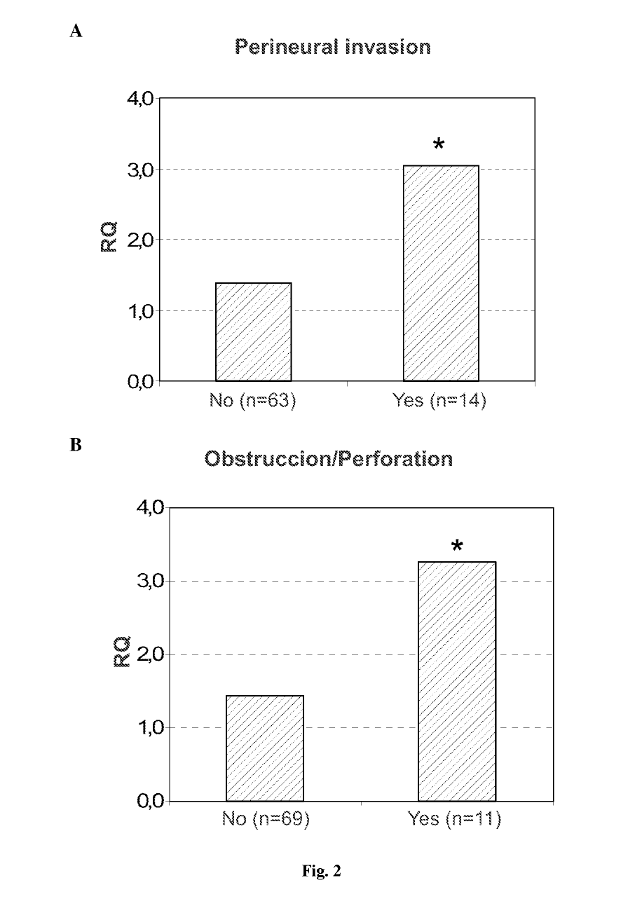

After the analysis of correlation between the available clinicopathological parameters and the expression levels of KIAA1456 in the clinical samples, it was found that there was a statistically significant correlation with perineural invasion (p=0.004) and intestinal obstruction syndrome (p=0.003) (FIGS. 2A and 2B, respectively).

The presence of perineural invasion corresponds to a category of factors with a prognostic value that has still not been sufficiently studied so as to be able to establish that they have said value.

There is a variety of analyses about the prognostic value of neoplastic intestinal obstruction where the authors assert that obstruction is the best clinical predictor with a reduction of the long-term survival [Ratio C. et al; Diseases of the Colon and Rectum 1998; 41:1033-1049]. In our series of patients we confirmed these data observing a statistical significance between neoplastic obstruction and a worse prognosis (data not shown).

### Example 3

### Analysis of the Prognostic Value of KIAA1456 Expression in Stage II Patients

### Analysis of the Prognostic Value of KIAA1456 Overexpression in Clinical Samples

It was tested whether KIAA1456 overexpression was relevant in the prognosis of the patient (FIG. 3). It was established as overexpression for RQ value over 1.5 (100% sensitivity, 81% specificity for overall survival and 95% sensitivity, 66% specificity for disease-free survival).

As shown in FIG. 3, a statistically significant association is observed between KIAA1456 overexpression and the recurrence of the patients measured as relapse-free survival (p=0.05).

### Analysis of the Prognostic Value of the Cases of Greater KIAA1456 Overexpression in Clinical Samples

It is known that certain patients with stage II disease have poorer prognosis. These are patients with at least one of the following clinical risk factors: T4 disease; tumor obstruction or perforation; poorly differentiated (grade 3) tumors; retrieval of <12 lymph nodes (ASCO recommendations); high preoperative CEA levels; vascular, lymphatic, and perineural invasion; and positive surgical margins in accordance with the College of American Pathologists consensus statement. Regarding these factors, it was found that patients with absence of vascular (FIGS. 4 A and C) or perineural invasion (Figure B and D), i.e. cases with much better prognosis, showed a significant decrease in relapse-free survival if they overexpressed KIAA1456 (FIG. 4), suggesting that KIAA1456 is correlated with a worse clinical outcome even when standard risk factors indicates a good prognosis.

Therefore, all these data demonstrate that KIAA1456 gene isoform 1 overexpression indicates a worse prognosis of stage II patients, proposing this gene as a prognostic marker in stage II CRC.

### Example 4

### Study of the Predictive Value of KIAA1456 Expression in Stage II CRC Patients

The results obtained in the previous sections indicate that KIAA1456 could work as a prognostic marker in these patients. However, what is more interesting for the patients of this stage, as has already been indicated, is the identification of predictive factors which allow selecting patients who will be more sensitive to the treatment of a conventional adjuvant chemotherapy, obtaining a therapeutic effect improving the survival thereof. Since this type of response prediction factor is still not available, there are no consistent selection criteria for adjuvant treatment in these patients with stage II colorectal cancer, and the need to administer chemotherapy or not is being highly questioned (J. Clin. Oncol. 1999, 17:1356-1363 and Mamounas et al.; J. Clin. Oncol. 1999, 17:1349-1355). It is estimated that the possible increase of survival which is achieved with chemotherapy after surgery compared to not administering chemotherapy is at best 2-4%.

Since information about recurrence and death of both patients who had been treated and patients who had not been treated was available in our series of patients, we wished to verify that the treatment with chemotherapy improved the survival of these patients.

Therefore, the following objective of the study consisted of stratifying the patients according to whether or not they had received adjuvant chemotherapy after surgery and conducting a study of the prognosis of these patients according to the KIAA1456 expression. The KIAA1456 expression cutoff points were chosen according to the significant overexpression for RQ values and the 69^{th} (overall survival), 64^{th} (disease-free survival) percentiles for the AQ values of KIAA1456.

As shown in FIG. 5 the prognosis of the patients who are not treated with adjuvant chemotherapy is radically worse in the patients with overexpression of the gene (p=0.01 for survival from colon cancer and p=0.012 for relapse-free survival) (FIGS. 5A and 5C). These differences are not observed in the patients treated with chemotherapy in which the prognosis is much better for both cases (FIGS. 5B and 5D).

These data were confirmed when using the cutoff point of the 69^{th} (overall survival) and 64^{th} (disease-free survival) percentiles for the AQ values of KIAA1456 (FIG. 6). Patients who are not treated showed both a significant less overall (p=0.03) and disease-free survival (p=0.025) when overexpressing KIAA1456. Again, these differences are not observed in patients treated with chemotherapy.

A multivariate analysis adjusted for sex, age, pathological grade, vascular and perineural invasion, obstruction/perforation showed KIAA1456 expression as a significant independent prognostic factor (HR 4.68, p=0.059 for relapse-free survival and HR 4.8, p=0.078 for overall survival).

These results demonstrate that KIAA1456 expression could be used as a predictive factor in patients with stage II CRC. This finding is useful because an accurate identification of the small percentage of 20-30% patients suffering stage II CRC which show relapse (Typically 20-30%) had not been possible until now.

1-16. (canceled)

17. A method for deciding on whether a therapy administered to a subject suffering from colorectal cancer is adequate, said method comprising determining the expression level of the KIAA1456 gene in a sample from said subject, wherein altered expression level of said gene when compared to a reference level is indicative that said therapy is adequate for said patient or wherein the same expression level of the KIAA1456 gene in said sample when compared to a reference level is indicative that said therapy is not adequate for said patient.

18. The method according to claim 17 wherein said altered expression level is either increased expression level or decreased expression level.

19. The method according to claim 17 wherein the sample is a tumor biopsy.

20. The method according to claim 17 wherein the expression level of the KIAA1456 gene is determined by measuring the level of mRNA encoded by the KIAA1456 gene or the level of KIAA1456 protein.

21. The method according to claim 20 wherein the mRNA level of the transcript variant 1 of the human KIAA1456 gene are determined.

22. The method according to claim 17 wherein patient is a human.

23. The method according to claim 17 wherein the patient has undergone surgical resection of the tumor.

24. The method according to claim 17 wherein the patient suffers from stage II colorectal cancer.

25. The method according to claim 17 wherein the patient does not show intestinal neoplasic obstruction, vascular invasion and/or perineural invasion.

26. A method for predicting the clinical outcome of a patient suffering from colorectal cancer comprising determining the expression level of the KIAA1456 gene in a sample from said patient wherein increased expression level of the KIAA1456 gene in said sample when compared with a reference level is indicative of a poor clinical outcome or wherein the same or decreased expression level of the KIAA1456 gene in said sample when compared with a reference level is indicative of a good clinical outcome.

27. The method according to claim 26 wherein the prediction of the clinical outcome is measured as disease-free survival and overall survival.

28. The method according to claim 26 wherein the patient has not been treated with neoadjuvant or adjuvant therapy.

29. The method according to claim 26 wherein the sample is a tumor biopsy.

30. The method according to claim 26 wherein the expression level of the KIAA1456 gene is determined by measuring the level of mRNA encoded by the KIAA1456 gene or the level of KIAA1456 protein.

31. The method according to claim 30 wherein the mRNA level of the transcript variant 1 of the human KIAA1456 gene is determined.

32. The method according to claim 26 wherein patient is a human.

33. The method according to claim 26 wherein the patient has undergone surgical resection of the tumor.

34. The method according to claim 26 wherein the patient suffers from stage II colorectal cancer.

35. The method according to claim 26, wherein the patient does not show intestinal neoplasic obstruction, vascular invasion and/or perineural invasion.

36. A kit comprising a set of reagents capable of specifically detecting the expression level of KIAA1456 and, optionally, a housekeeping gene or the protein encoded by said housekeeping gene wherein the reagents of the kit are DNA or RNA probes capable of specifically detecting the mRNA level of KIAA1456 and/or or antibodies capable of specifically detecting the level of KIAA1456 protein.

37. A method for deciding whether a therapy administered to a subject suffering from colorectal cancer is adequate or for predicting the clinical outcome of a patient suffering from colorectal cancer, said method comprising determining the expression level of KIAA1456 in a sample from said subject using the kit according to claim 36.
